(19) United States
(12) Patent Application Publication
(10) Pub. No.: US 2023/0248306 A1
(43) Pub. Date: Aug. 10, 2023

(54) WIRELESS, NON-INVASIVE, CONTINUOUS MONITORING OF CEREBROSPINAL FLUID FLOW THROUGH SHUNTS

(71) Applicant: RHAEOS, INC., Evanston, IL (US)

(72) Inventors: Siddharth KRISHNAN, Cambridge, MA (US); Adam ZYSK, Chicago, IL (US); Amit AYER, Palo Alto, CA (US); Anna Lisa SOMERA, Glenview, IL (US); Michelle HASSE, Elkhart, IL (US)

(21) Appl. No.: 18/003,218

(22) PCT Filed: Jun. 24, 2021
(86) PCT No.: PCT/US2021/038850

§ 371 Date: Dec. 23, 2022

Related U.S. Application Data
(60) Provisional application No. 63/043,720, filed on Jun. 24, 2020.

(51) Int. Cl. A61B 5/00; A61B 5/01; A61B 5/03; A61B 5/11

(52) U.S. Cl. CPC A61B 5/4851; A61B 5/01; A61B 5/031; A61B 5/6832; A61B 5/1114; A61B 5/742; A61B 5/7278; A61B 5/7264; A61B 2562/164; A61B 2562/0271; A61B 2562/0219; A61B 2560/0214; A61B 2562/043

(57) ABSTRACT

Systems and methods for monitoring flow of cerebrospinal fluid through shunts are disclosed. A wireless, flexible flow sensor may comprise a substrate, a thermal actuation mechanism configured to supply thermal energy to a portion of a skin surface of the body, the portion of the skin surface overlaying a subdermal conduit for a body fluid, a temperature sensor configured to detect a change in a temperature related to the portion of the skin surface, a motion sensor supported by the substrate and configured to detect an orientation related to a segment of the subdermal conduit, and a microprocessor in wireless communication with a controller. The microprocessor may comprise circuitry configured to receive, from the controller, a first signal to activate the thermal actuation mechanism; and receive, from the temperature sensor, a second signal associated with the change in temperature related to the portion of the skin surface.

### CROSS REFERENCE TO RELATED APPLICATIONS

This application is based on and claims benefit of priority of U.S. Provisional Patent Application No. 63/043,720, the contents of which are incorporated herein by reference in its entirety.

### TECHNICAL FIELD

The description herein generally relates to the field of cerebrospinal fluid shunts, and more particularly to systems and methods of monitoring and measuring flow of cerebrospinal fluid in shunts using non-invasive, wearable epidermal electronics. This invention was made with government support under NSF Award 1938472 awarded by National Science Foundation (NSF). The government has certain rights in the invention.

### BACKGROUND

Hydrocephalus is a common and costly condition caused by the accumulation of cerebrospinal fluid (CSF) in the brain, with symptoms that include headaches, seizures, coma, or death. It particularly afflicts children, occurring in 1-5 of every 1,000 live births. Hydrocephalus is usually treated with the surgical implantation of a catheter, known as a ventricular shunt, which diverts the excess CSF in the brain to a distal absorptive site, such as the peritoneal cavity. Overall, 125,000 shunts are implanted or replaced annually in United States, with costs estimated at $2 billion per year. Unfortunately, shunts have extremely high failure rates due to a diverse set of factors including occlusion, mispositioning, or kinking. Up to 51% of shunt recipients exhibit symptoms that require a shunt repair surgery, known as a “revision,” in the first year and 99% of shunts are replaced at least once within 10 years. It is also estimated that nearly half of all healthcare expenditures related to shunt surgery are spent on revisions. Non-specific symptoms like headaches and nausea make diagnosing shunt malfunction extremely challenging. Consequently, physicians rely on a complicated, expensive, and often inconclusive suite of tests, including those that expose patients to significant radiation, to make clinical decisions.

On average, a single pediatric hydrocephalus patient with an implanted shunt is admitted to the hospital multiple times each year, with a high number of emergency department presentations. The standard-of-care methods used to assess shunt malfunction today include (i) aspiratory CSF sampling, an invasive procedure used to assess pressure in the shunt, (ii) injection of a radionucleotide tracer into the ventricular system and monitoring the movement of the radiotracer into the abdomen, (iii) an x-ray “shunt series” exam to check for mechanical shunt damage, and (iv) Computerized Tomography (CT) or Magnetic Resonance Imaging (MRI) to assess ventricle size. Notably, CT and x-ray shunt series expose the patient to significant radiation. Also, the only test that directly measures CSF flow in the shunt, the radiotracer test, is invasive, painful, and carries a risk of infection. Moreover, the diagnostic performance of these tests is poor. X-ray shunt series, for example, has a sensitivity of approximately 18% for the determination of shunt failure in symptomatic patients. Finally, MRI and CT have poor performance because shunt failure is often not concomitant to ventriculomegaly. These tests are regularly employed despite their serious limitations because they are, unfortunately, the best tools currently available to clinicians.

In addition to their poor performance, frequent CTs expose children to sizeable radiation doses, increasing their risk of brain cancer. Although MRI is seen as a viable alternative to CT for evaluation of hydrocephalus, access and cost are significant barriers. Significantly, only 17% of the CTs performed lead to a revision surgery within a week, implying that the vast majority of these scans are unnecessary.

Therefore, the current standard of care, ventricular catheters (shunts), is prone to failure, which can result in nonspecific symptoms such as headaches, dizziness, and nausea. Current diagnostic tools for shunt failure such as CT, MRI, radionuclide shunt patency studies (RSPSs), and ice pack-mediated thermodilution have disadvantages including high cost, poor accuracy, inconvenience, and safety concerns. As an example, ShuntCheck® utilizes an ice-pack based thermal cooling system connected to a Windows PC DAQ to address a need for shunt monitoring. That technology, however, is cumbersome and time-consuming. The device's cumbersome, multi-step protocol; equivocal or negative past clinical studies; and need for ice-pack cooling have limited its acceptance. Additionally, patient discomfort due to prolonged skin cooling (detrimental for pediatric diagnostics) and absence of chronic monitoring further limits its diagnostic relevance. Accordingly, there is a need for a precise, rapid, easy-to-use, wireless, non-invasive shunt diagnostic, that is conformable to skin and has epidermal-like mechanical properties.

Intracranial pressure (ICP) is defined as the pressure inside the skull, and therefore, the pressure inside the brain tissue and the cerebrospinal fluid (CSF). Because the brain is incompressible, when the skull is intact, the sum of the volumes of brain, CSF, and intracranial blood is constant. Normal ICP varies with age and body posture but is generally considered to be 5-15 mmHg in healthy supine adults, 3-7 mmHg in children and 1.5-6 mmHg in infants. ICP>20 mmHg is considered to be elevated, and this is considered an important cause of secondary injury leading to irreversible brain injury and sometimes death. ICP monitoring is used in a number of conditions including, but not limited to traumatic brain injury, intracerebral hemorrhage, subarachnoid hemorrhage, hydrocephalus, malignant infarction, cerebral edema, CNS infections, hepatic encephalopathy, to name a few, and in all of these conditions ICP monitoring in the light of other parameters can influence management for better outcomes.

A ventriculoperitoneal (VP) shunt assembly is typically used to remove the excess CSF from the brain. Ventriculoperitoneal shunt assemblies may comprise a proximal catheter, a distal catheter, and a pressure regulated valve that connects the proximal and the distal catheters. The valves in a VP shunt assembly may be factory-calibrated to relate ICP with the flow of the fluid. Some commercially available valve models (e.g., Strata, Medtronic Inc.) are adjustable and rely on externalized magnets to perform non-invasive changes to valve settings that are routinely performed on clinical patients. Knowledge of accurate flow rates and valve setting may allow for direct determination of ICP. Accordingly, quantitative measurements of flow rates of CSF may be desirable.

There are several conditions where it may be important to monitor ICP, as even minor fluctuations may require a change in management. The gold standard for monitoring ICP is an intraventricular catheter connected to an external pressure transducer; the catheter is placed into one of the ventricles through a burr hole. The catheter can also be used for therapeutic CSF drainage and for administration of drugs. Even though it remains an accurate and cost-effective method of ICP monitoring, it is associated with a number of complications. These include risk of infection, hemorrhage, obstruction, difficulty in placement, malposition, etc. Other invasive modalities for ICP monitoring, all of which entail the same complications as intraventricular catheter insertion, include intraparenchymal monitors, subdural, and epidural devices, as well as lumbar puncture measurements. Accordingly, there is a need for systems and methods of determining the ICP accurately and reliably using non-invasive techniques.

Furthermore, other desirable features and characteristics will become apparent from the subsequent detailed description and the appended claims, taken in conjunction with the accompanying drawings and this background of the disclosure.

### SUMMARY

Embodiments of the present disclosure provide systems and methods of monitoring flow of cerebrospinal fluid through shunts. Disclosed herein are exemplary systems and methods of measurements of flow using a flexible, non-invasive device gently laminated onto the surface of the skin at the location of the shunt. The results presented here extend these concepts into a user-friendly, fully wireless system that enables continuous, noninvasive monitoring of CSF flow performed by patients themselves in real-world settings. Advanced designs and integration schemes exploit low-cost commercial components and flexible circuit board manufacturing techniques in optimized layouts guided by theoretical and numerical models of thermal transport and system-level mechanics. A Bluetooth Low-Energy System on a Chip (BLE-SoC) embedded system architecture allows for robust, high-quality data transfer during normal patient activities, where a miniaturized on-board, rechargeable battery supports continuous operation for several hours. On-body measurements and field trials on hydrocephalus patients reveal reliable operation during both short ‘spot-checks’ and, for the first time, extended measurements of flow during natural motions of the body and for different orientations. The results suggest broad applicability for monitoring of shunts in patients across age ranges, pathologies, and settings, including the home.

In one aspect of the disclosure, a wireless, flexible flow sensor mountable on a body is disclosed. The sensor may comprise a substrate, a thermal actuation mechanism supported by the substrate and configured to supply thermal energy to a portion of a skin surface of the body, the portion of the skin surface overlaying a subdermal conduit for a body fluid, a temperature sensor supported by the substrate and configured to detect a change in a temperature related to the portion of the skin surface, a microprocessor in wireless communication with a controller, comprising circuitry configured to: receive, from the controller, a first signal to activate the thermal actuation mechanism; and receive, from the temperature sensor, a second signal associated with the change in temperature related to the portion of the skin surface, and a power source configured to supply electrical power to at least one of the thermal actuation mechanism, the temperature sensor, and the microprocessor.

In another aspect of the disclosure, a wireless, flexible flow sensor mountable on a body is disclosed. The sensory may comprise a substrate, a thermal actuation mechanism supported by the substrate and configured to supply thermal energy to a portion of a skin surface of the body, the portion of the skin surface overlaying a subdermal conduit for a body fluid, a temperature sensor supported by the substrate and configured to detect a change in a temperature related to the portion of the skin surface, a motion sensor supported by the substrate and configured to detect an orientation related to a segment of the subdermal conduit, a motion sensor supported by the substrate and configured to detect an orientation related to a segment of the subdermal conduit, a microprocessor in wireless communication with a controller, comprising circuitry configured to: receive, from the controller, a first signal to activate the thermal actuation mechanism from the controller; and receive, from the temperature sensor, a second signal associated with the change in temperature related to the portion of the skin surface, and a power source configured to supply electrical power to at least one of the thermal actuation mechanism, the temperature sensor, and the microprocessor. The motion sensor may comprise an accelerometer, a gyroscope, a magnetometer, a three-axis digital accelerometer.

In another aspect of the disclosure, a wireless fluid flow monitoring system is disclosed. The system may comprise a flexible flow sensor mountable on a body. The flow sensor may comprise a temperature sensor configured to continuously detect a change in temperature related to a portion of a skin surface of the body, the portion of the skin surface overlaying a subdermal conduit for a body fluid, a power source configured to supply electrical power to at least one of the thermal actuation mechanism and the temperature sensor, a receiving circuit in electrical communication with the power source, wherein the receiving circuit is configured to receive electromagnetic energy. The system may further include a power charging unit configured to wirelessly transmit electromagnetic energy to a receiver of the receiving circuit; and a processor in wireless communication with the power charging unit and the flexible flow sensor, the processor configured to receive, from the flexible flow sensor, data associated with the change in temperature related to the portion of the skin surface of the body; determine a flow rate of the body fluid through the segment of the subdermal conduit based on the received data; store, in a database, the received data and the determined flow rate of the body fluid.

In yet another aspect of the disclosure, a method of continuous flow measurement of a body fluid using a wireless, flexible flow sensor comprising a motion sensor is disclosed. The method may include sending, to a user-device for display, an indication to mount the flow sensor on a portion of a skin surface of a body, the portion of the skin surface overlaying a subdermal conduit of the body fluid; detecting, using the motion sensor, a first position of the body; sending, to the user-device for display, a second indication to adjust a position of the body to a second position of the body different from the first position; detecting, using the motion sensor, the second position of the body; and determining a change in flow related to the body fluid through a segment of the subdermal conduit, corresponding to a change in related to the position of the body.

In yet another aspect of the present disclosure, a computer-implemented system for continuously determining flow rate of a body fluid through a subdermal conduit is disclosed. The system may comprise a memory storing instructions, and a processor configured to execute the instructions to receive, from a temperature sensor of a flexible flow sensor, information associated with a temperature related to a portion of a skin surface, the portion of the skin surface overlaying the subdermal conduit, and a time of temperature measurement, the temperature sensor comprising: a plurality of upstream temperature sensors configured to detect an upstream temperature related to the portion of the skin surface upstream of a thermal actuator of the flexible flow sensor; a plurality of downstream temperature sensors configured to detect a downstream temperature related to the portion of the skin surface downstream of a thermal actuator of the flexible flow sensor. The processor may be further configured to execute instructions to receive, from the motion sensor, information associated with an orientation of a segment of the subdermal conduit and the time of temperature measurement; compute a first value indicating a difference between the upstream and the downstream temperatures and a second value indicating an average of the upstream and downstream temperatures; and determine the flow rate of the body fluid based on a comparison of finite element analysis and the computed first and second values.

In yet another aspect of the disclosure, a method of measuring intracranial pressure (ICP) using a wireless flexible flow sensor is disclosed. The method may include applying, using a thermal actuator of the flow sensor assembly, heat to a portion of a surface of a skin overlaying a subdermal conduit of the body fluid; measuring, using a first thermal sensor of the flow sensor assembly, a first temperature of the body fluid flowing upstream of the thermal actuator; measuring, using a second thermal sensor of the flow sensor assembly, a second temperature of the body fluid flowing downstream of the thermal actuator; determining a first flow rate related to the body fluid through a segment of the subdermal conduit based on a difference between the first and the second temperature of the body fluid; determining a second flow rate related to the body fluid based on the first flow rate and a characteristic of the skin; and determining intracranial pressure based on the determined second flow rate.

### BRIEF DESCRIPTION OF THE DRAWINGS

FIG. 1 is a schematic illustration of an exemplary on-shunt and off-shunt positions of a wireless flow sensor, consistent with disclosed embodiments.

FIG. 2A is a schematic illustration of an exploded view of the wireless flow sensor of FIG. 1, consistent with disclosed embodiments.

FIG. 2B is a schematic illustration of electronic components of the wireless flow sensor of FIG. 1, consistent with disclosed embodiments.

FIG. 2C shows optical images of an exemplary application for wireless readout and communication with the wireless flow sensor, consistent with disclosed embodiments.

FIG. 3 is a schematic illustration of an exemplary wireless flow sensor, consistent with disclosed embodiments.

FIG. 4 is a schematic illustration of components of the exemplary wireless flow sensor shown in FIG. 3, consistent with disclosed embodiments.

FIG. 5 is a schematic illustration of an exemplary bench-top model of an exemplary wireless flow sensor on a shunt embedded in silicone skin phantom, consistent with disclosed embodiments.

FIGS. 6A and 6B illustrate a schematic of a side view and a top view infra-red (IR) thermograph, respectively, of an exemplary flow sensor mounted above an underlying shunt indicating a “no-flow” scenario, consistent with disclosed embodiments.

FIGS. 6C and 6D illustrate a schematic of a side view and a top view infra-red (IR) thermograph, respectively, of an exemplary flow sensor mounted above an underlying shunt indicating a “flow” and a flow direction of a body fluid, consistent with disclosed embodiments.

FIG. 7 illustrates a data graph of temperature differential (a) based on the position of a flow sensor with respect to an underlying shunt, consistent with disclosed embodiments.

FIG. 8A illustrates data graphs of temperature differential (a) through a shunt based on the body position of the subject over a period of time, consistent with disclosed embodiments.

FIG. 8B illustrates data graphs of temperature differential (a) averaged over time at on-shunt locations, consistent with disclosed embodiments.

FIGS. 9A and 9B illustrate data graphs of temperature differential (a) of a subject descending and ascending an elevator, respectively, measured at on-shunt locations, consistent with disclosed embodiments.

FIGS. 10A-10C illustrate data graphs of temperature differential (a) measured for extended time-periods, consistent with disclosed embodiments.

FIGS. 11A-11D illustrate data graphs of rotational tolerance measured at different angles associated with misplacement of an exemplary wireless flow sensor, consistent with disclosed embodiments.

FIGS. 12A-12D illustrate data graphs of translational tolerance measured at different distances associated with misplacement of an exemplary wireless flow sensor, consistent with disclosed embodiments.

FIGS. 13A-13D illustrate data graphs of improvement of signal to noise ratio (SNR) with incorporation of a thermal insulation in an exemplary wireless flow sensor, consistent with disclosed embodiments.

FIGS. 14A and 14B illustrate flow rate measurements in a shunt using an exemplary wireless flow sensor, consistent with disclosed embodiments.

FIGS. 15A-15E illustrate simulated and measured extended flow rate measurements in a shunt using an exemplary wireless flow sensor, consistent with disclosed embodiments.

FIG. 16 is a block diagram illustrating components of an exemplary wireless flow monitoring system, consistent with disclosed embodiments.

FIGS. 17A and 17B are schematic illustrations of exemplary relative orientations of a thermal actuator and analog-to-digital converters (ADCs) in a wireless flow sensing system of FIG. 16, consistent with disclosed embodiments.

FIG. 18 illustrates a flow chart showing an exemplary method of measuring flow rates of a body fluid through a shunt, consistent with disclosed embodiments.

FIGS. 19A and 19B illustrate representative calibration charts for conversion of raw data based on a model, consistent with disclosed embodiments.

FIG. 20 is a schematic illustration of an exemplary calibration chart for a commercially available ventriculoperitoneal shunt assembly valve.

FIG. 21 is a schematic illustration of an exemplary chart showing the range, median and average values of CSF flow rate.

FIG. 22 illustrates a data graph comparing the modeling and experimental values of the transient response of thermal anisotropy, consistent with disclosed embodiments.

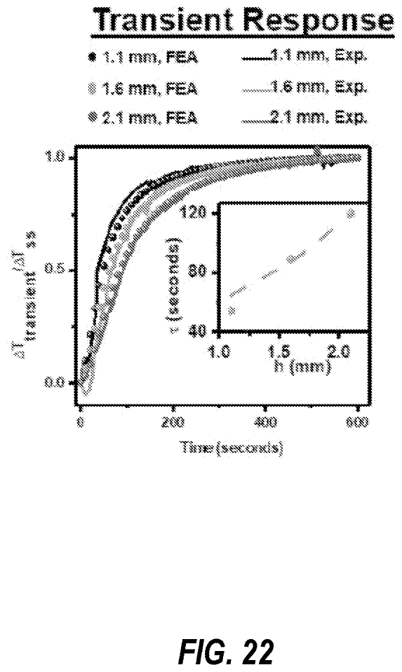

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the disclosure. Instead, they are merely examples of apparatuses and methods consistent with aspects related to the subject matter recited in the appended claims. The following detailed description refers to the accompanying drawings. While several illustrative embodiments are described herein, modifications, adaptations and other implementations are possible. For example, substitutions, additions, or modifications may be made to the components and steps illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope of the invention is defined by the appended claims.

Relative dimensions of components in drawings may be exaggerated for clarity. Within the following description of drawings, the same or like reference numbers refer to the same or like components or entities, and only the differences with respect to the individual embodiments are described.

As used herein, unless specifically stated otherwise, the term “or” encompasses all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

In general, the terms and phrases used herein have their art-recognized meaning, which can be found by reference to standard texts, journal references and contexts known to those skilled in the art. The following definitions are provided to clarify their specific use in the context of the invention.

“Soft” refers to a material that may be comfortably positioned against the skin without discomfort or irritation to the underlying skin by the material itself deforming to conform to the skin without unduly exerting force on the underlying skin with corresponding device-generated skin deformation. Softness/hardness may be optionally quantified, such as in terms of durometer, or a material's resistance to deformation. For example, the substrate may be characterized in terms of a Shore 00 hardness scale, such as a Shore 00 that is less than 80. Soft may also be characterized in terms of a modulus, such as a Young's modulus that is less than or equal to 100 kPa.

“Stretchable” refers to a material's ability to undergo reversible deformation under an applied strain. This may be characterized by a Young's modulus, a ratio of stress to strain. A bulk or effective Young's modulus refers to a composite material formed from materials having different Young's modulus, so that the bulk or effective Young's modulus is influenced by each of the different materials and provides an overall device-level modulus.

“Flexible” refers to a material's ability to undergo a bending with fracture or permanent deformation, and may be described in terms of a bending modulus.

Any of the devices may be described herein as being “mechanically matched” to skin, specifically the skin over which the device will rest. This matching of device to skin refers to a conformable interface, for example, useful for establishing conformal contact with the surface of the tissue. Devices and methods may incorporate mechanically functional substrates comprising soft materials, for example exhibiting flexibility and/or stretchability, such as polymeric and/or elastomeric materials. A mechanically matched substrate may have a modulus less than or equal to 100 MP a, less than or equal to 10 MPa, less than or equal to 1 MPa A mechanically matched substrate may have a thickness less than or equal to 0.5 mm, and optionally for some embodiments, less than or equal to 1 cm, and optionally for some embodiments, less than or equal to 3 mm. A mechanically matched substrate may have a bending stiffness less than or equal to 1 nN m, optionally less than or equal to 0.5 nN m.

A mechanically matched device, and more particularly a substrate is characterized by one or more mechanical properties and/or physical properties that are within a specified factor of the same parameter for an epidermal layer of the skin, such as a factor of 10 or a factor of 2. For example, a substrate may have a Young's Modulus or thickness that is within a factor of 20, or optionally for some applications within a factor of 10, or optionally for some applications within a factor of 2, of a tissue, such as an epidermal layer of the skin, at the interface with a device of the present invention. A mechanically matched substrate may have a mass or modulus that is equal to or lower than that of skin.

“Encapsulate” refers to the orientation of one structure such that it is at least partially, and in some cases completely, surrounded by one or more other structures, such as a substrate, adhesive layer or encapsulating layer. “Partially encapsulated” refers to the orientation of one structure such that it is partially surrounded by one or more other structures, for example, wherein 30%, or optionally 50%, or optionally 90% of the external surface of the structure is surrounded by one or more structures. “Completely encapsulated” refers to the orientation of one structure such that it is completely surrounded by one or more other structures.

“Polymer” refers to a macromolecule composed of repeating structural units connected by covalent chemical bonds or the polymerization product of one or more monomers, often characterized by a high molecular weight. The term polymer includes homopolymers, or polymers consisting essentially of a single repeating monomer subunit. The term polymer also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers useable in the methods, devices and components disclosed include, but are not limited to, plastics, elastomers, thermoplastic elastomers, elasto-plastics, thermoplastics and acrylates. Exemplary polymers include, but are not limited to, acetal polymers, biodegradable polymers, cellulosic polymers, fluoropolymers, nylons, polvacrylonitrile polymers, polyamide-imide polymers, polyimides, polyacrylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

“Elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to silicon containing polymers such as polysiloxanes including poly(dimethyl siloxane) (i.e. PDMS and h-PDMS), poly(methylsiloxane), partially alkylated poly(methyl siloxane), poly(alkyl methyl siloxane) and poly(phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a polymer is an elastomer.

“Conformable” refers to a device, material or substrate which has a bending stiffness that is sufficiently low to allow the device, material or substrate to adopt any desired contour profile, for example a contour profile allowing for conformal contact with a surface having a pattern of relief features. In certain embodiments, a desired contour profile is that of skin.

“Conformal contact” refers to contact established between a device and a receiving surface, specifically skin. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In an embodiment, conformal contact involves adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. Devices of certain aspects are capable of establishing conformal contact with internal and external tissue. Devices of certain aspects are capable of establishing conformal contact with tissue surfaces characterized by a range of surface morphologies including planar, curved, contoured, macro-featured and micro-featured surfaces and any combination of these. Devices of certain aspects are capable of establishing conformal contact with tissue surfaces corresponding to tissue undergoing movement.

“Young's modulus” is a mechanical property of a material, device or layer which refers to the ratio of stress to strain for a given substance. “Low modulus” refers to materials having a Young's modulus less than or equal to 10 MPa, less than or equal to 5 MPa or less than or equal to 1 MPa.

“Bending stiffness” is a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

“Thermal actuation state” refers to the thermal actuator that is on an off-state or an on-state. In this context. “substantially independent” refers to a position of the reference sensor that is sufficiently separated from the actuator that the reference sensor output is independent of whether the thermal actuator is on or off. Of course, the systems and methods presented herein are compatible with relatively minor effects of the actuator on the reference sensor, such as within 5%, within 1% or within 0.1% of a reference temperature when the actuator is in the on state compared to when the actuator is in the off state. Depending on specific device and tissue characteristics, this distance may be between about 10 mm and 20 mm, such as about 15 mm.

Ventricular shunts represent an essential component of clinical treatment for hydrocephalus, a common and debilitating neurological disorder that results from the overproduction and/or impaired reabsorption of CSF produced in the ventricular system of the brain. Hydrocephalus arises from a number of causes, including but not limited to cancer, hemorrhage, trauma, and congenital malformations. This condition affects an estimated 750,000 patients in the United States alone, and it is responsible for ˜3.1% of all pediatric acute care costs. Approximately 125,000 pediatric hydrocephalus patients in the US account for 400,000 days spent in the hospital each year. Shunts assemblies typically involve two silicone catheters, connected upstream and downstream of a regulating valve, to drain excess CSF from the ventricle to a distal absorptive site, usually the peritoneum, pleura, or right atrium of the heart. While effective in CSF diversion and prevention of the sequelae of hydrocephalus, including seizures, coma, neurological injury and death, shunts are highly prone to failure due to fibrinous catheter ingrowth, kinking, discontinuity, over-drainage, distal malabsorption and infection, among other conditions. An estimated 84.5% shunt recipients require revision operations. Clinical symptoms of shunt malfunction tend to be non-specific, such as headache, nausea and somnolence, thereby creating challenges in clinical diagnosis. Because ramifications of misdiagnosis can include severe morbidity and mortality, isolating the location and cause of failure may be beneficial in the appropriate care of hydrocephalic patients.

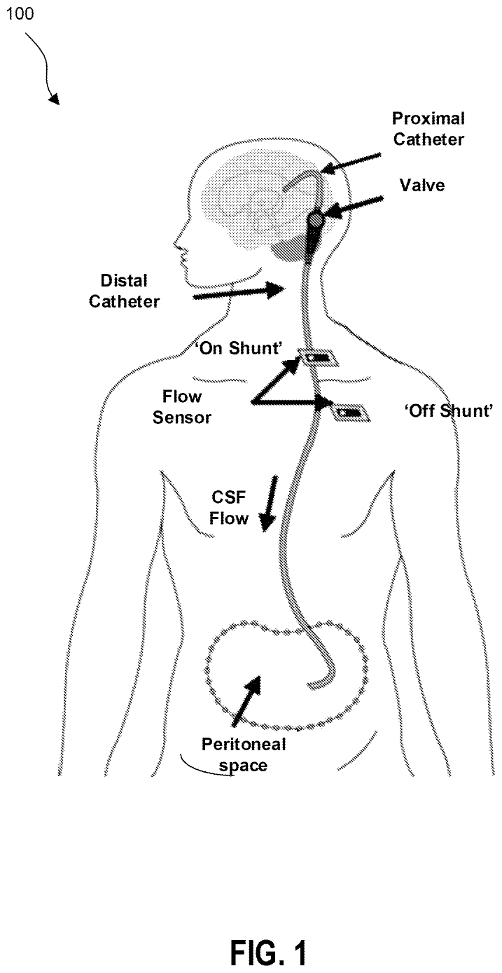

Reference is now made to FIG. 1, which illustrates an anatomy 100 of a typical CSF shunt assembly and “on-shunt” and “off-shunt” positions of a wireless flow sensor. The most commonly used treatment for hydrocephalus is diversion of CSF from the ventricles to the peritoneal cavity by means of a permanent prosthetic shunt such as a CSF-VP shunt (Ventricular-Peritoneal), as illustrated in FIG. 1. A CSF shunt may comprise a valve connected to a tube. The proximal end of the tube, also referred to herein as proximal catheter, is surgically inserted into the ventricle of the brain, and runs subcutaneously through the body into the abdominal or the peritoneal cavity. In the context of this disclosure, “proximal” refers to the area, region, or section closer to the brain, and “distal” refers to the area, region, or section farther from the brain. Under normal operation, the direction of CSF flow may be from the ventricular area to the peritoneal area or from the proximal catheter to the distal catheter, the flow of CSF regulated by a valve. A wireless, flexible, non-invasive flow sensor may be placed on a skin surface of a patient's body overlaying the shunt, with a mild adhesive where the shunt is most superficial, typically near the neck or the clavicle. Although beneficial, the location of the flow sensor may not be restricted to the clavicle. In some embodiments, the flow sensor may be positioned anywhere along the catheter, as appropriate, or based on the other factors such as patient comfort, skin thickness, availability and accessibility of the flow sensor, among other things.

Measurements on human subjects involve placement of the flow sensor at a distal location along the shunt above the clavicle, referred to herein as “on-shunt”, guided by visual examination and tactile feel, among other things. In some embodiments, alignment marks on the device and temporary markings on the skin, formed with a surgical pen, may facilitate alignment and positioning. An additional measurement at a location of the skin adjacent to the shunt but devoid of near-surface vasculature, referred to herein as “off-shunt” may serve as a control spot, representing the ‘zero-flow’ case, as illustrated in FIG. 1. In some embodiments, a handheld ultrasound instrument may be used to capture images of the skin and the underlying shunt tubing. In some preferred embodiments, the distance from the top outer surface of the shunt tubing to the surface of the skin may be 1.4 mm. Typically, locations where the shunt is easily palpated are typically <2 mm from the surface of the skin.

In some embodiments, devices disclosed herein may comprise a miniaturized (<5 mm diameter) thermal actuator configured to deliver small, precisely controlled thermal power (<5 mW/mm²) to the surface of the skin, thereby creating an imperceptible local increase in temperature (˜5 K). When positioned at the location of a shunt, the directionality and the magnitude of the flow of CSF may affect the resulting distribution of temperature at the surface of the skin. The increases in temperature downstream (Tts) from the actuator may be larger than those at an equal distance upstream (T_{US}). Temperature sensors may be installed to record these differences as a function of time after supplying power to the actuator. Quantitative values of flow rate can be determined from these data using multi-physics computational models that include the essential geometric parameters of the integrated system (shunt, device, and skin) and constitutive properties of the materials.

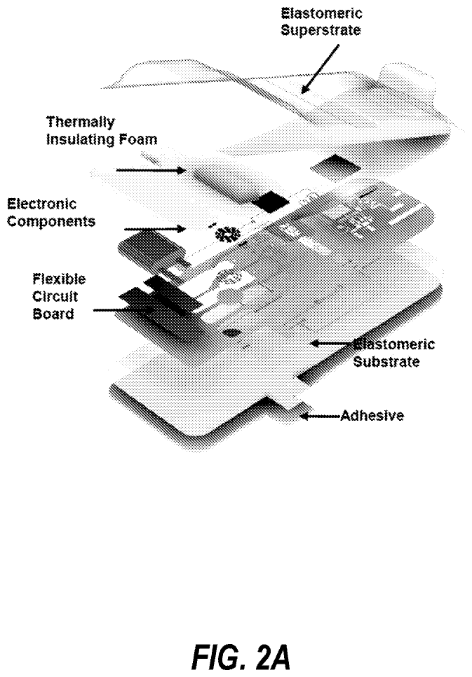

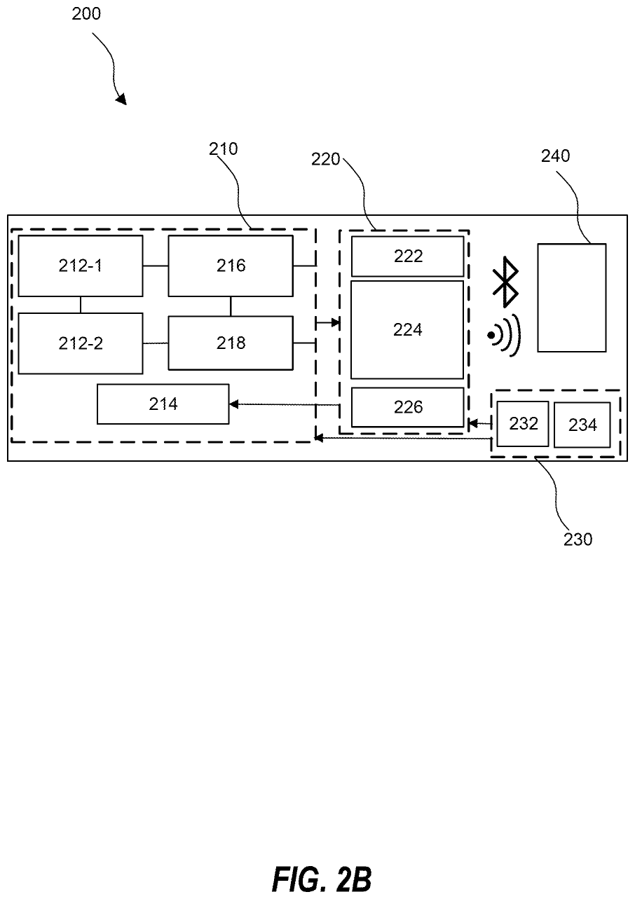

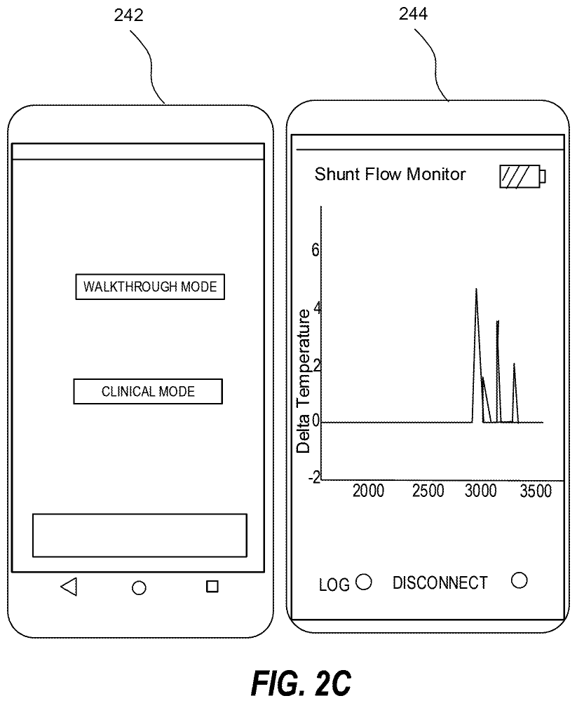

Reference is now made to FIG. 2A, which illustrates an exploded view of an exemplary flow sensor device, consistent with disclosed embodiments. In some embodiments, the flow sensor may comprise a layered structure comprising an elastomeric substrate, a flexible printed circuit board (fPCB) substrate to support and connect the electronic components, a thermal insulation layer, and an elastomeric superstrate. The flow sensor may further comprise an adhesive layer configured to provide adhesion between the skin and the elastomeric substrate as well as between the elastomeric substrate and the fPCB substrate disposed on the elastomeric substrate.

In some embodiments, the fPCB has a thickness ˜115 μm that yields low flexural rigidity (4×10⁻⁴ N-m) and sufficient degrees of flexibility to conform and bond to the curved surface of the skin with a mild adhesive where the shunt is most superficial, typically near the neck or the clavicle. This follows from an island-bridge configuration, designed to localize bending strains to the interconnected structures and away from the electronic components. The result may (i) facilitate conformal contact with the skin while reducing potential for delamination and (ii) minimize strain on the rigid electronic components and soldered interfaces between the components and the fPCB. Mechanical finite element analysis (FEA) results (not shown) indicate that the strains on the interconnect layer remain low (<1%) during routine bending associated with mounting on the neck for children (radius of curvature ˜40 mm) and adults (55 mm).

Under normal operation and disposition of the flow sensor, the fPCB may comprise planar areas and non-planar areas. The planar areas may support electronic components that connect through thin, mechanically stable conductive traces on the fPCB. A soft, low-modulus silicone elastomer (Young's modulus E˜60 kPa) may encapsulate substantially the entire system, as illustrated in FIG. 2A. Several considerations may inform the choice of mechanics, materials, and form factor, including adhesion, comfort, safety, and thermal transport. In some embodiments, a thin, soft silicone layer of ˜100 μm thickness, and E=1.4 MPa, on the underside of the device may substantially cover the skin-facing side of the fPCB. The ability to establish strong, yet repeatable and non-irritating contact with the skin may represent a key consideration, facilitated by the thin, soft construction of the device and by a medical-grade, double-sided acrylate-silicone adhesive. The high adhesion energy of the acrylate layer (˜350 N/m) may establish strong contact to the flow sensor, while the comparatively low adhesion of the silicone layer (˜33 N/m) may form a gentle interface to the skin while maintaining excellent thermal coupling. In some embodiments, the flow sensor may further comprise a liner-release layer with a custom laser-structured tab which may facilitate handling and mounting/dismounting. Peeling back the tab may expose the adhesive for mounting on the skin. The entire silicone and double-sided adhesive assembly may have a thickness of less than 500 μm, for example ˜120 μm and may add a thermal mass of 14 mJ/cm²-K, equivalent to that of a <50 μm thick layer of skin. In some embodiments, the total thickness of silicone and double-sided adhesive assembly may be more than 500 μm. In some embodiments, the total thickness of silicone and double-sided adhesive assembly may be in a range from 40-2000 μm, or 40-1500 μm, or 40-1000 μm, or 40-500 μm, or 40-400 μm, or 40-300 μm, or 40-200 μm, or 40-100 μm, or other thickness range, as appropriate.

In some embodiments, the flow sensor may be a flexible sensor placed on a subject's skin. Although, the radius of curvature on a human neck region may be 70 mm or less, the flow sensor may be bent to larger radii of curvatures, while maintaining its operability and integrity.

Reference is now made to FIG. 2B, which illustrates a block diagram of the electronic components of the flow sensor system 200, consistent with disclosed embodiments. One or more of these components may be supported by the fPCB such that the components may be interconnected, if so desired. The components may include, but are not limited to, thermal sensing and actuating components, analog front-end circuitry to convert resistance measurements of temperature into corresponding output voltages, a BLE-SoC (Bluetooth Low Energy System on Chip) and its associated timers and antenna to digitize and transmit these data, and also to support wireless two-way communication, power management electronics to supply power to the various sub-systems. The power management electronics may include a rechargeable lithium polymer (Li—Po) battery, a power management circuit, and an inductive coil for wireless recharging of the battery. The fully wireless design of the proposed flow sensor represents a key advance over existing technologies by allowing the patient to move freely while generating a continuous stream of data transmitted directly to a hand-held electronic device including, but not limited to, a smartphone, a tablet, a computer, a laptop, or any suitable electronic device having a graphical user interface (GUI) for displaying the data.

As an example, as illustrated in FIG. 2B, flow sensor system 200 may comprise analog front-end circuitry 210, a BLE-SoC (Bluetooth Low Energy System on Chip) 220, a power management module 230, and a graphic user-interface 240. In an exemplary flow sensor system, analog front-end circuitry 210 may include temperature sensors 212-1 and 212-2, an actuator 214, active H-bridges 216 and 218. In some embodiments, BLE-SoC 220 may include antennae 222 and 224, and an input/output module (GP I/O) 226. In some embodiments, power management module 230 may include power management circuitry 232 and a power source 234. Graphic user-interface 240 may include, but is not limited to, a smartphone, a tablet, a computer, a laptop, a hand-held device, or any suitable electronic device configurable to receive, transmit, store, upload, download, display data and further communicate with a wired or a wireless network.

Reference is now made to FIG. 2C, which illustrates an exemplary software application enabled device, consistent with disclosed embodiments. A custom software application may serve as a control interface, as well as a means to record, store and display data on any BLE-enabled device (smartphone, tablet, etc.). The software may also provide step-by-step on-screen instructions to guide users on the operating procedures. As an example, display 242 on graphic-user interface 240 may allow a user to select an operation mode such as, for example, a walkthrough mode or a clinical mode. Display 244 on graphic user-interface 240 may display data to the user in a selectable representation including, but not limited to, graphical, tabular, textual, etc. A toggle-switch on the user interface may control the operation of a resistive thermal actuator, with power provided through the BLE-SoC. An analog front-end circuit based on an active Wheatstone bridge network may be configured to convert data from resistive temperature sensing elements into voltages. The BLE-SoC may be configured to digitize and transmit these data to the BLE-enabled device, where they can be analyzed to yield rates of CSF flow.

In some embodiments, the flow sensor may be configured to continuously monitor the flow of CSF through the CSF-VP shunt. Continuous flow measurements on freely moving patients represents a fundamentally new mode for monitoring. The utility of the flow sensor reaches beyond performing simple binary assessments of flow/no-flow to establishing correlations between dynamic, real-time changes and patient sensations/activities. For example, changes in body orientation can affect flow, as suggested by patient complaints of headaches when lying down or immediately after standing up. In a healthy, asymptomatic outpatient, measurements before, during, and after changes in body orientation may suggest corresponding changes in flow. As an example, an otherwise healthy asymptomatic outpatient described changes in position from upright to leaning forward (45°) as a case for headaches, for example, during reading. Results from continuous monitoring on this patient show that leaning both forward and backward (45°) leads to an instantaneous and significant reduction in flow that coincided with headaches. Reversal of flow, as indicated by a negative value of a, was also seen.

In some embodiments, though not illustrated in FIGS. 2A-2C, the flow sensor may comprise an accelerometer. The accelerometer may be configured to detect changes in position, acceleration, or orientation of the subject's body relative to the CSF shunt. The change in positioning may be accompanied by a reduction in flow, and in some cases, a mild backflow. The accelerometer may comprise a single or a multi-axis accelerometer. In preferred embodiments, the flow sensor may include a three-axis digital accelerometer compatible with the integration techniques of the flow sensor. The three-axis digital accelerometer may have high characteristics such as sampling frequency, a 16-bit resolution, a broad bandwidth response, and a wide dynamic range, among other suitable characteristics.

In some embodiments, the accelerometry data may be time-stamped. For example, the positional, orientational, or the acceleration data of the subject may be time-stamped such that it may be associated with the change in flow rate through the CSF shunt, thereby allowing analysis of the impact of patient positioning on the flow rate of the CSF.

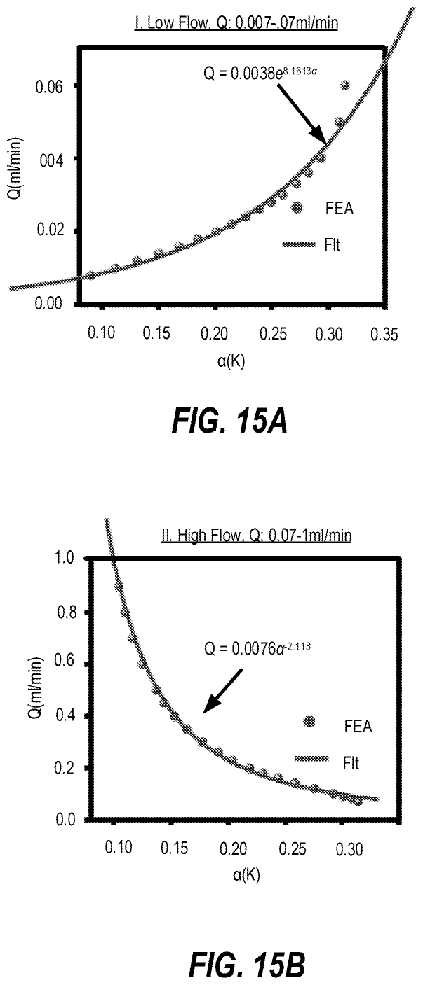

In some embodiments, the wireless flow sensor may comprise a thermal insulation layer. The thermal insulation layer may be a thermally insulating polyurethane foam disposed over the sensing and actuating elements. Disposing the thermal insulation layer over the thermal actuator and sensors may improve the signal-to-noise ratio (SNR), as illustrated in FIG. 13, and may fundamentally enable an aspect of the design disclosed herein by significantly reducing sources of noise that can be induced by air flow. This effect can be understood by considering the rate of heat transfer from the negative temperature coefficient (NTC) sensor to its surroundings by free-convection and its linear dependence on the convective heat transfer coefficient, H_{free}. The side-wall of the NTC can be modeled as a simple vertical plate, and accordingly, H_{free} is

$${H}_{free} = \frac{{k}_{air} {CRa}_{L}^{n}}{L} \qquad ( 1 )$$

where L is the height of the NTC element (˜300 μm), n and C are empirical fitting factors known to be 0.25 and 0.59 respectively for laminar flows and Ra_{L} is the Rayleigh number for free convection across L, given by

$$R {a}_{L} = \frac{g β ( {T}_{NTC} - {T}_{∞} ) {L}^{3}}{v ς} \qquad ( 2 )$$

where g is the acceleration due to gravity, β is the volumetric fluid expansion coefficient of air, ν is the thermal diffusivity of air and ζ is the kinematic viscosity of air. T_{NTC} and T_{∞} are the temperature of the NTC and its surroundings, respectively. The addition of a foam layer effectively prevents air circulation around the NTC, and therefore free-convection effects across the vertical surface of the NTC, with a magnitude that is only weakly dependent on foam formulation and pore size, for materials examined here, above a critical thickness of ˜1 mm.

To quantify flow-induced thermal anisotropy, consider the parameter a (T_{DS}−T_{US}) as the difference between the average temperature determined by the downstream and the upstream NTC sensors, where T_{US} and T_{DS} represent changes in temperature from a steady-state baseline value prior to actuation. In the absence of flow, α is ˜0 K and in its presence, α>0, with a value that is typically >30 times larger than the noise for practical scenarios of relevance to hydrocephalus patients. The temporal response of α to a change in flow is a function of the thermal mass of the fPCB assembly, including the NTCs and the actuator, and the characteristic diffusion time associated with thermal transport through underlying skin to the shunt, t_{diffusion}˜hₛₖᵢₙ²/νₛₖᵢₙ.

The thickness of the skin and/or underlying subcutaneous fat layers that lies over the shunt, hₛₖᵢₙ may also strongly influences α. As hₛₖᵢₙ increases, the sensitivity may decrease. Though the sensitivity is insufficient for hₛₖᵢₙ larger than ˜4 mm, this limitation is not expected to be relevant for measurements over the neck/clavicle region where hₛₖᵢₙ is typically between 0.5 mm and 2 mm. To distinguish between flow rates associated with identical values of α on either side of its peak value, a second parameter, β≡(T_{DS}+T_{US})/2, the average change in temperature may be considered. T_{DS} may vary non-monotonically with flow, increasing with flow for 0<Q<0.07 ml/min and decreasing with flow for Q>0.07 ml/min. By contrast, T_{US} may decrease with flow for a full range of flow rates. As a result, their average, β, is relatively constant at low flow rates and decreases with flow at high rates for steady-state measurements. In effect, β is a measure of the increased net thermal transport (i.e, non-directional) due to convection, and decreases monotonically with flow rate. As a result, β can be used to yield information about flow regime (high vs. low) while α can serve as a measure of flow rate. Combining these two parameters allows for the determination of flow rate, as discussed later in this disclosure.

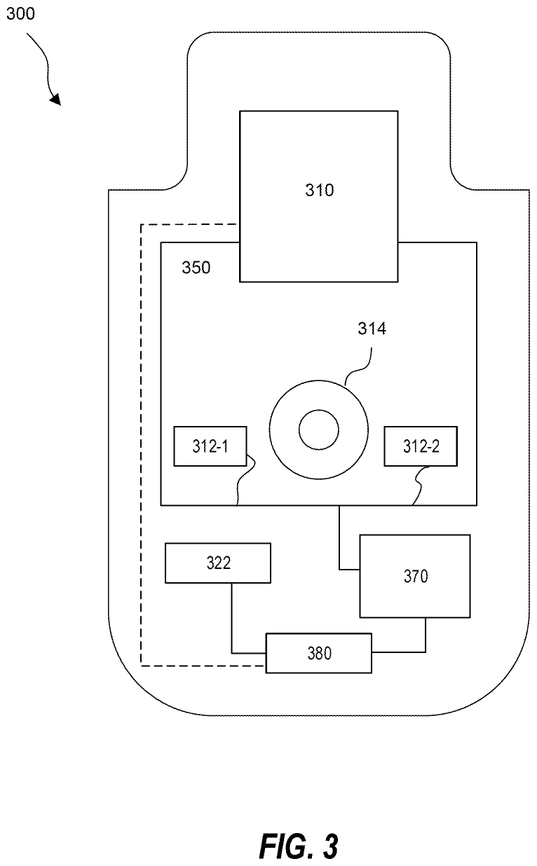

Reference is now made to FIG. 3, which illustrates a schematic representation of an exemplary wireless flow sensor 3M), consistent with some disclosed embodiments. Flow sensor 300 may include, but is not limited to, a power source 310 (e.g., a Li-polymer battery), a substrate 350, an actuator or an actuation mechanism 314, temperature sensors 312-1 and 312-2, an antenna 322, a processor 370, and a regulator 380. It is to be appreciated that although not illustrated, wireless flow sensor 300 may include more actuators, temperature sensors, regulators, processors, antennae, power sources, etc. Further, although not shown, wireless flow sensor 300 may include more or fewer components desirable to perform the functions, as appropriate. In some embodiments, wireless flow sensor 300, also referred to herein as an epidermal linear array (ELA), may include soft, conformal thermal sensing and actuating components, a flexible PCB (fPCB) substrate, surface mounted electronic components such as antenna, regulators, etc.

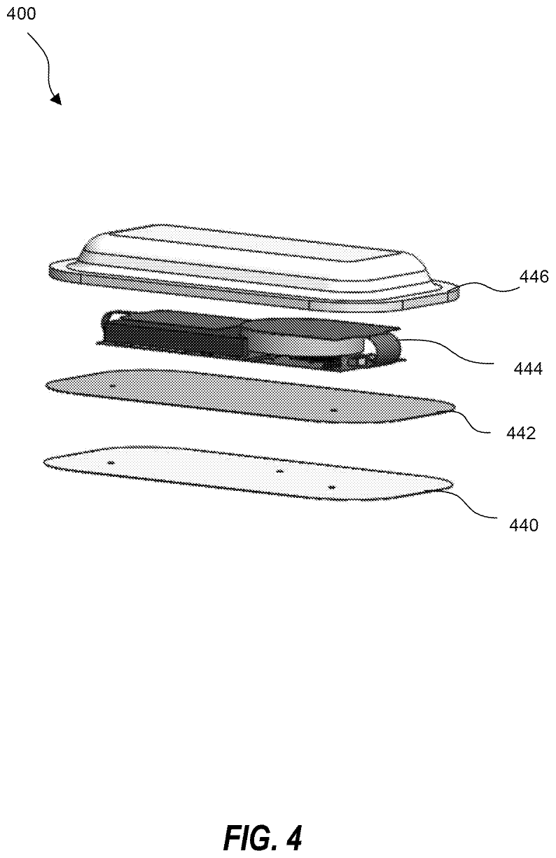

FIG. 4 illustrates an exploded view of an exemplary wireless flow sensor 400 comprising multiple layers. The wireless flow sensor 400 may include a patient adhesive laminate 440, a housing adhesive layer 442, a flexible circuit board 444, a housing 446. It is to be appreciated that these are exemplary designs and wireless flow sensor may comprise other components, not illustrated or described, relevant for performing the desired functions.

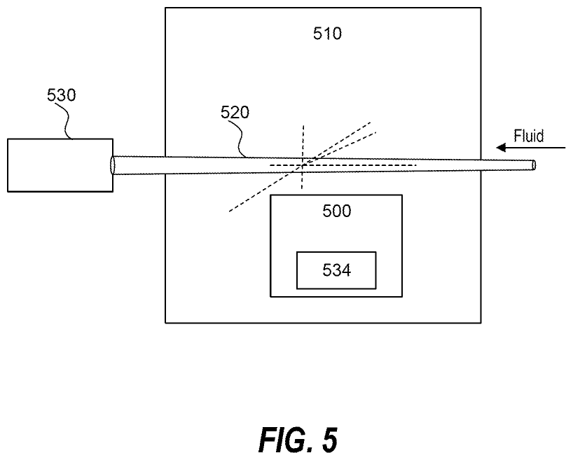

Reference is now made to FIG. 5, which illustrates a benchtop model system comprising a phantom skin model for simulation of the flow rates, skin thicknesses and skin thermal properties relevant to measurements of CSF flow through shunts, consistent with disclosed embodiments. The benchtop model system may comprise a distal catheter 520 (OD=2.1 mm, ID=1.1 mm) of a VP shunt assembly embedded inside a silicone skin phantom 510, at a depth of 1.1 mm. An exemplary silicone skin phantom may comprise a well-mixed combination of polydimethylsiloxane (PDMS) and PDMS doped with carbon black microparticles. Other combinations of materials may be used to make skin phantoms, as appropriate. Different ratios of the two materials yielded a range values of kₛₖᵢₙ relevant to the stratum corneum (k_{SC}˜0.25 W/m-K), epidermis (k_{epidermis}˜0.35 W/m-K) and subcutaneous fat layer (k_{SC}˜0.2 W/m-K). Spin-casting and laminating silicone sheets of different thicknesses (0.5 mm, 1.0 mm, 2.0 mm, 3.0 mm, 4.0 mm) and mixing ratios onto the phantom assembly simulated the desired values of hₛₖᵢₙ and kₛₖᵢₙ. A calibrated syringe pump (not illustrated) connected to the distal catheter through a VP shunt valve 530 may be configured to supply flow through the assembly. Water served as the test fluid, as it forms 99% of CSF. A commercially available medical-grade adhesive may be used to bond a wireless flow sensor 500 comprising a power source 534 (e.g., a battery) to the phantom assembly. In some embodiments, a 3-D printed mold may allowed for the generation of skin layers of varying thicknesses to simulate the effects of different skin mounting locations on patients. The benchtop assembly may be used to explore the effects of (i) skin thickness, (ii) ambient convection, (iii) skin thermal properties, (iv) near-surface blood vessels, (v) motion artifacts, and (vi) thermal actuation power, to establish a set of operational parameters for the device, and to establish bounds on reliable operation.

Experimental validation followed two protocols. Protocol 1 simulated step-changes in flow to measure real-time sensitivity and time dynamics. Mounting the device on the skin phantom assembly with no flow (i.e, 0 ml/min) allowed it to thermally equilibrate with the surface temperature for 120 s. Following this equilibration period, operation of the actuator resulted in a local increase in temperature of <5K over 180 s. Flow began at t=180 s after actuation, for an additional 180 s. At t=360 s after actuation, flow ended, and the temperatures re-equilibrated for a final 180 s. Conducting this experiment for two flows rates, 0.05 ml/min and 0.5 ml/min, bounded the responses expected for the full range of healthy flow rates. The time intervals used in these tests exceeded the natural response times of wireless flow sensor 500, syringe pump, and skin phantom 510.

Protocol 2 simulated constant flow during a 5-min measurement period, to establish values of T_{DS}, T_{US}, α and β for steady-state flow conditions, as a scenario of direct relevance to on-body patient trials. Here, flow at a pre-determined rate initially equilibrated for a period of 60 s. Laminating the device onto the skin phantom over the shunt for 120 s allowed the system to thermally equilibrate, as in protocol 1. Operating the actuator and collecting data from the 4 NTCs for a period of 300 s completed the protocol. We conducted this protocol for the following flow rates: Q=0 ml/min, 0.03 ml/min, 0.05 ml/min, 0.07 ml/min, 0.1 ml/min, 0.2 ml/min, 0.3 ml/min, 0.5 ml/min, 0.7 ml/min. Temperature calibrations allowed conversion of changes in resistances of the negative temperature coefficients (NTCs) to temperature measurements.

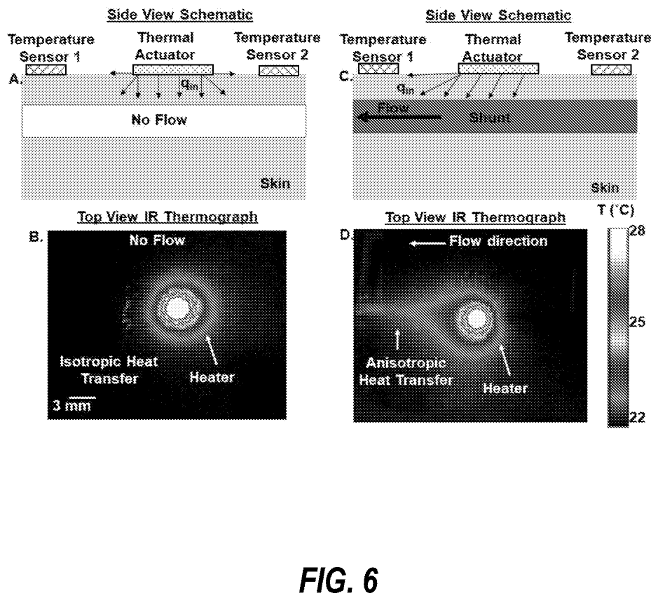

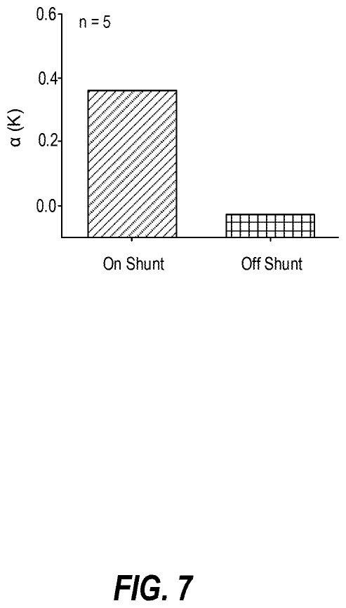

Reference is now made to FIGS. 6A-6D, which illustrate schematics and side views of the epidermal flow sensing based on the anisotropy of thermal power dissipation in presence of CSF flow, consistent with disclosed embodiments. A flow sensor device may be placed on a skin surface of the subject (patient), as shown in the side view of the device mounted above an underlaid non-flowing shunt or a flowing shunt. As shown in FIGS. 6C and 6D, when the thermal actuator is mounted on the skin directly overlaying a source of biological flow, such as through a superficial blood vessel or a shunt, the thermal power is dissipated anisotropically and preferentially in the direction of the flowing fluid. Conversely, on skin devoid of underlying flow conduits, as shown in FIGS. 6A and 6B, the thermal power may be transmitted isotropically. These effects can be captured by precise temperature sensors located adjacent to the actuator, with the resulting temperature measurements serving as a direct correlate for flow. In some embodiments, the system may comprise upstream temperature sensors and downstream temperature sensors to measure the temperature of the fluid upstream and downstream of the thermal actuator. In the context of this disclosure, upstream of the thermal actuator refers to the portion between the thermal actuator and the valve and proximal to the ventricle, and downstream of the thermal actuator refers to the portion between the thermal actuator and the peritoneal cavity and distal from the ventricle.

In the absence of flow, or at a suitable ‘off-shunt’ location such as the base of the pectoral muscle immediately distal to the clavicle, measurements from the temperature sensors increase smoothly monotonically with values that are nearly the same to within 50 mK resulting in root-mean square (RMS) values of αRMS˜13 mK with peak-to-peak variations of 8 mK, across a 100 s averaging window (representing the thermal response time). Similar measurements over the shunt display clear thermal anisotropy, with αRMS˜250 mK (75 mK) and peak-to-peak variations of 60 mK (25 mK) for high flow (low flow) cases. These data establish that values of α>50 mK are above the noise level and can be assumed to result from flow. The observations also reveal that the transient period of the response has a duration of ˜100 s.

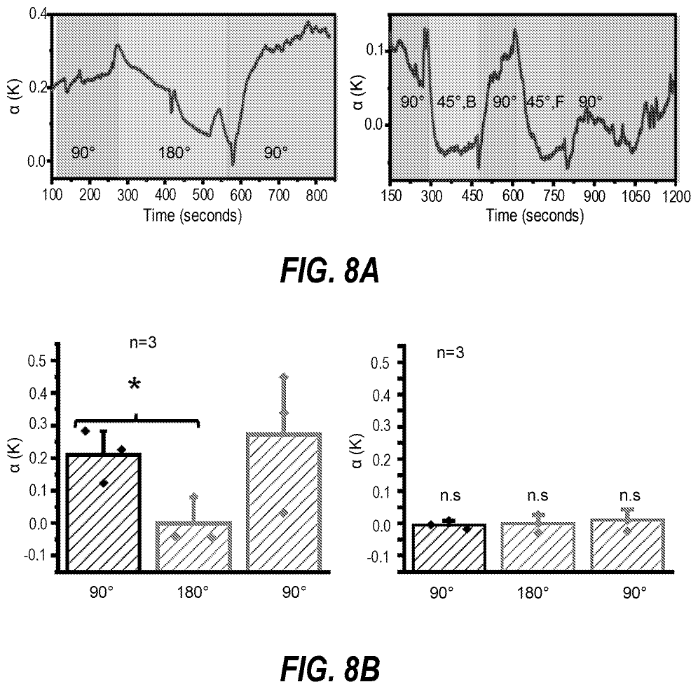

Reference is now made to FIG. 7, which illustrates a data graph of temperature differential (a) based on the position of a flow sensor with respect to an underlying shunt, consistent with disclosed embodiments.

Evaluations on additional patients establish the repeatability and robustness of operation across a range of age groups and pathologies without suspected shunt malfunction. In some embodiments, a smartphone with the software application, or a GUI (e.g., graphic user-interface 240 of FIG. 2) may be operated by an attending or resident care provider and configured to receive temperature data from the device and plots the results in real-time on a graphical user interface. The wireless interface may allow the physician freedom of motion, within a range of ˜6 m from the bed of the patient, without loss of substantial connection or aberrant signals. Similarly, the patients can also move freely in and around their hospital bed without disruption in wireless connectivity or motion induced artifacts in the measurement. In all cases, on-shunt measurements averaged over 100 s after the transient 100 s period (α_{on shunt}=0.35±0.14 K) differ (p=0.003) from off-shunt measurements (α_{off shunt}=−0.03±0.02 K) in a paired student-t test. These results demonstrate high levels of performance and data reliability largely because the wireless embodiment may minimize motion-induced partial delamination and strain-induced electrical noise, and the thermal insulation foam may isolate the measurement system from ambient thermal fluctuations and time-variant convective effects.

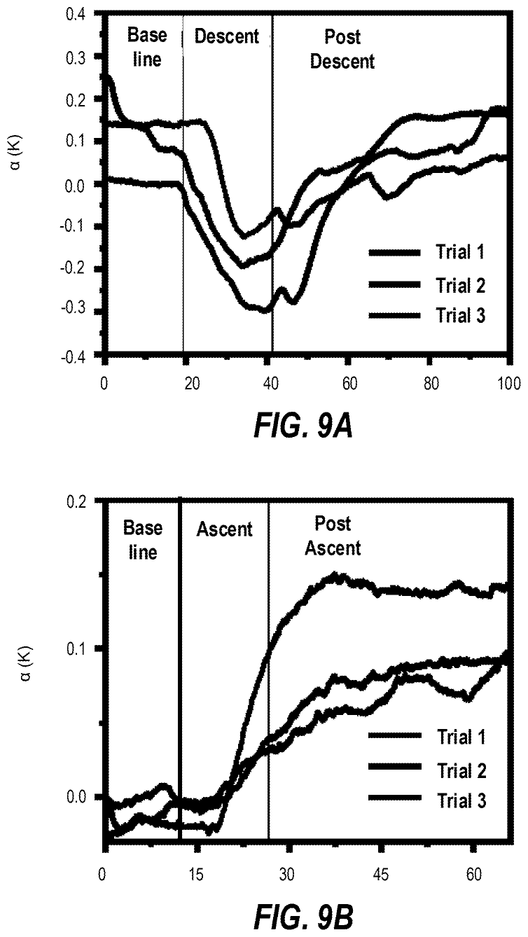

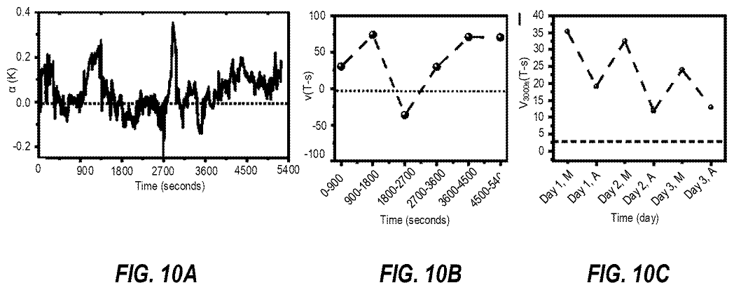

Reference is now made to FIGS. 8A-8B, which illustrate data graphs of temperature differential (α) through a shunt based on the body position of the subject over a period of time, consistent with disclosed embodiments. The temperature differential measurements may include measurement of temperature difference between the downstream and the upstream temperatures (T_{DS}−T_{US}) as a function of patient positioning. The temperature difference measurements may be correlated with the flow rate, as discussed later in this disclosure. Continuous flow measurements on freely moving patients may represent a fundamentally new mode for monitoring. Here, the utility of the device reaches beyond performing simple binary assessments of flow/no-flow to establishing correlates between dynamic, real-time changes and patient sensations/activities. For example, changes in body orientation can affect flow, as suggested by patient complaints of headaches when lying down or immediately after standing up. In a healthy, asymptomatic outpatient, measurements before, during, and after changes in body orientation suggest corresponding changes in flow, as illustrated in FIG. 8A. Initially, measurements performed with the patient sitting upright (90°) yield values consistent with a normal, healthy daytime flow at a constant rate (α˜0.2-0.3 K). Reclining to a supine position (180°) lead to a gradual decline of flow over ˜200 s, consistent with other findings performed with externalized drains and in-dwelling shunts with flows measured with radioactive tracers and ultra-sound imaging with air-bubbles introduced into the shunt, until the volunteer returns to a sitting upright position (90°), where the measurements indicate a return to baseline flow rates. Additional tests on two healthy, asymptomatic outpatients indicated similar results with clear (p=0.04) differences between measurements during an initial upright period and a subsequent supine period as computed for a paired t-test. Control measurements off-shunt showed no thermal anisotropy, independent of body orientation, as expected.

Patients can often identify instances of aberrant flow based on the onset of characteristic headaches. In some instances, an otherwise healthy asymptomatic outpatient described changes in position from upright to leaning forward (45°) as a case for headaches, for example, during reading. Results from continuous monitoring on this patient show that leaning both forward and backward by an angle of 45° may lead to an instantaneous and significant reduction in flow that coincided with headaches. Reversal of flow, as indicated by a negative value of a, was also observed. In both cases, flow appears to recover back to a positive, baseline value, though at different rates.

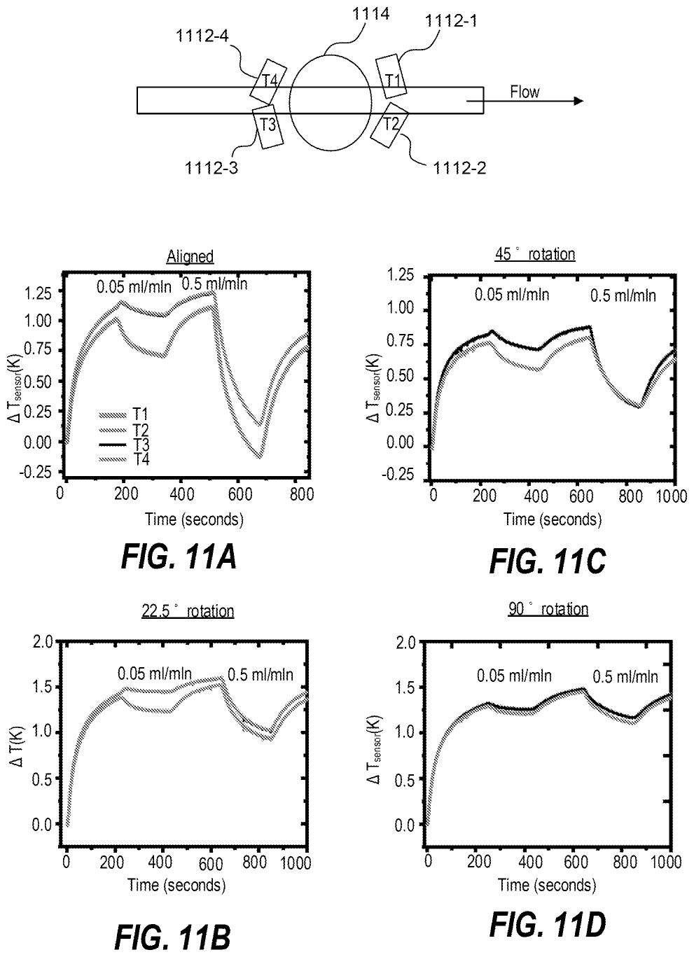

In some embodiments, an otherwise healthy, outpatient complained of headaches during rapid inertial changes associated with riding on elevators in high-rise buildings. Measurements on this patient performed during three routine elevator ascents and descents reveal characteristics consistent with corresponding changes in flow during the periods of acceleration, where descent reduces flow, to the level of back-flow and ascent enhances flow, as illustrated in FIGS. 9A-9B.

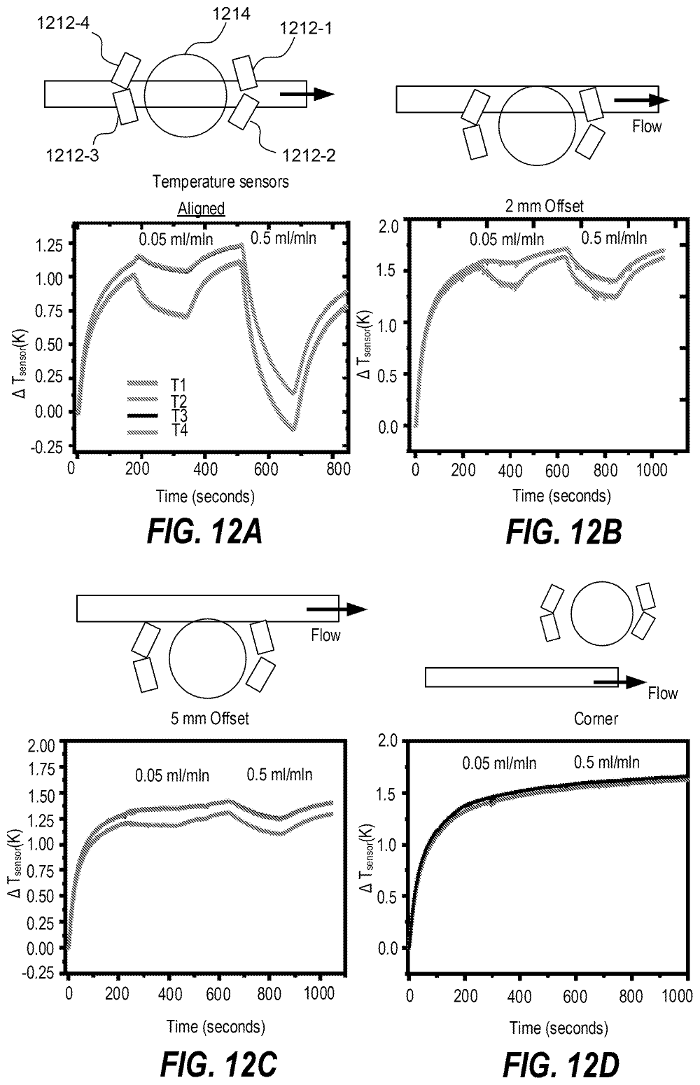

Reference is now made to FIGS. 10A-10C, which illustrate measurements of the temperature differential (α) between downstream and upstream temperature sensors for extended time periods, consistent with disclosed embodiments. In addition to the episodic changes described in FIGS. 8A-8B and 9A-9B, variations can be measured over a longer time course, either through continuous monitoring or by comparison of repeated measurements. Continuous monitoring of an outpatient for a period of ˜1.5 h, during normal behaviors, with the acquisition smartphone placed in a pocket, illustrates these capabilities. These data demonstrate intermittent flow with time scales (˜20 min) that are consistent with prior data collected on patients with externalized ventricular drains. Total CSF volume extracted during shunt taps serves as an important diagnostic measure of patient health, and time integration of α(t) yields a parameter. γ, that serves as a correlate for total volumetric flow during a fixed time interval. Such integrated measurements for 15-min. time intervals may highlight changes in flow output over the monitoring period as an indicator of shunt intermittency. On longer time-scales, γ values can also serve as points of comparison across days or longer. In the context of this disclosure, continuous measurement may refer to uninterrupted measurements for 20 minutes or more, 30 minutes or more, 40 minutes or more, 60 minutes or more, 90 minutes or more, 120 minutes or more, or 240 minutes or more. In some embodiments, the duration of measurement may be limited by the power available in the power source of the flow sensor to operate one or more of the thermal actuators, thermal sensors, receivers, transmitters, or other sub-systems requiring power to function.

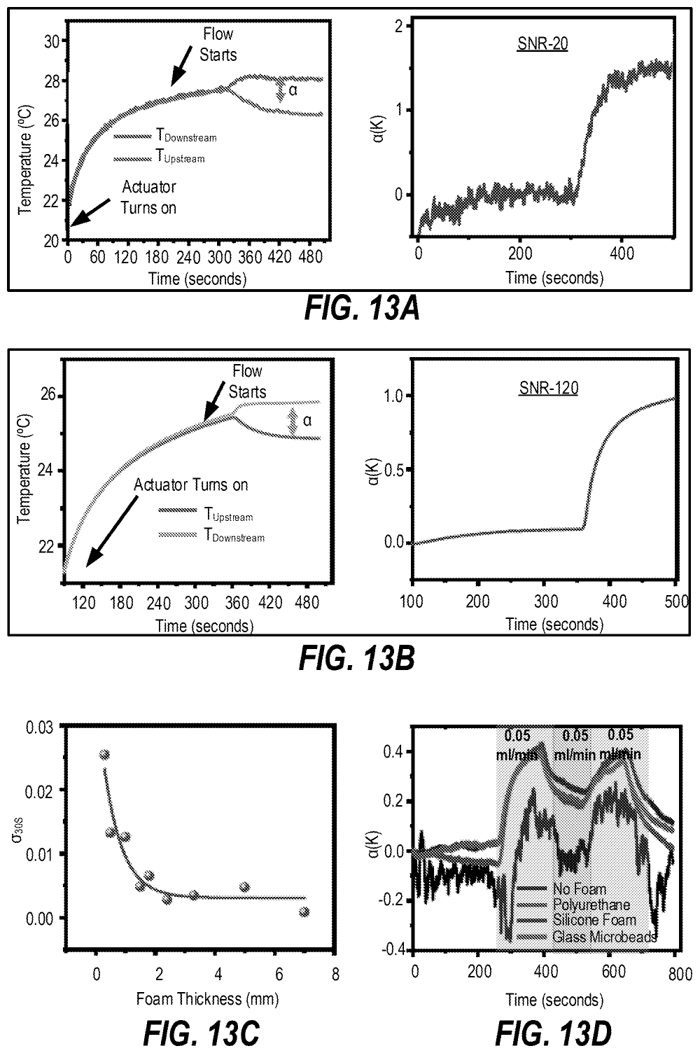

Reference is now made to FIGS. 11A-D, which illustrate data graphs of rotational tolerance measured at different angles associated with misplacement of an exemplary wireless flow sensor measured on benchtop shunt assembly for two flow rates relevant to physiological CSF flow, consistent with disclosed embodiments. As illustrated, the benchtop shunt assembly may comprise at least an actuator 1114 and temperature sensors 1112-1-1112-4 surrounding actuator 1114. FIG. 11A represents the temperature difference measured over a time period when the wireless flow sensor is aligned with respect to the shunt. FIG. 11B represents the temperature difference measured over a time period when the wireless flow sensor is placed at 22.5° with respect to the fluid through the shunt. FIG. 11C represents the temperature difference measured over a time period when the wireless flow sensor is placed at 450 with respect to the fluid through the shunt. FIG. 11D represents the temperature difference measured over a time period when the wireless flow sensor is placed at 90° with respect to the fluid through the shunt.

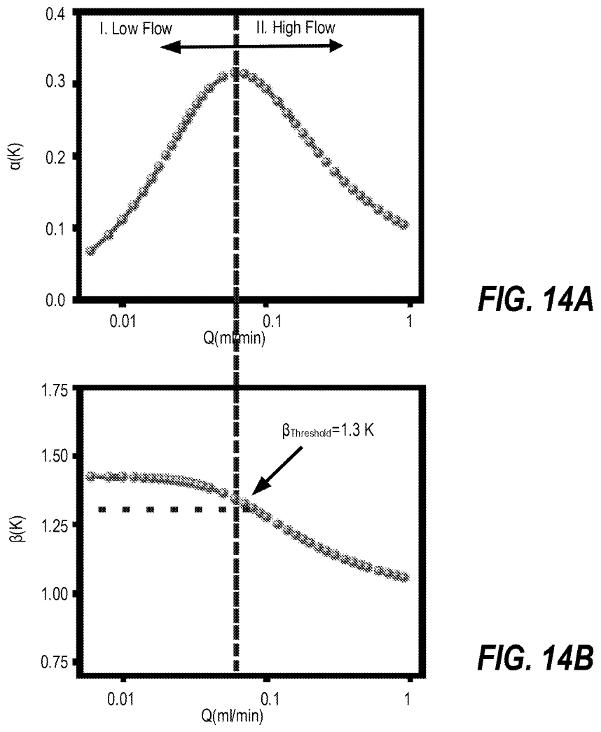

FIGS. 12A-D illustrate data graphs of translational tolerance measured at different distances associated with misplacement of an exemplary wireless flow sensor for two flow rates relevant to physiological CSF flow, consistent with disclosed embodiments. As illustrated, the benchtop shunt assembly may comprise an actuator 1214 and temperature sensors 1212-1-1112-4 surrounding actuator 1214. FIG. 12A represents the temperature difference measured over a time period when the wireless flow sensor is aligned with respect to the shunt. FIG. 12B represents the temperature difference measured over a time period when the wireless flow sensor is placed at a 2 mm offset with respect to the shunt. FIG. 12C represents the temperature difference measured over a time period when the wireless flow sensor is placed at a 5 mm offset with respect to the shunt. FIG. 12D represents the temperature difference measured over a time period when the wireless flow sensor is placed at an offset greater than 5 mm with respect to the shunt.

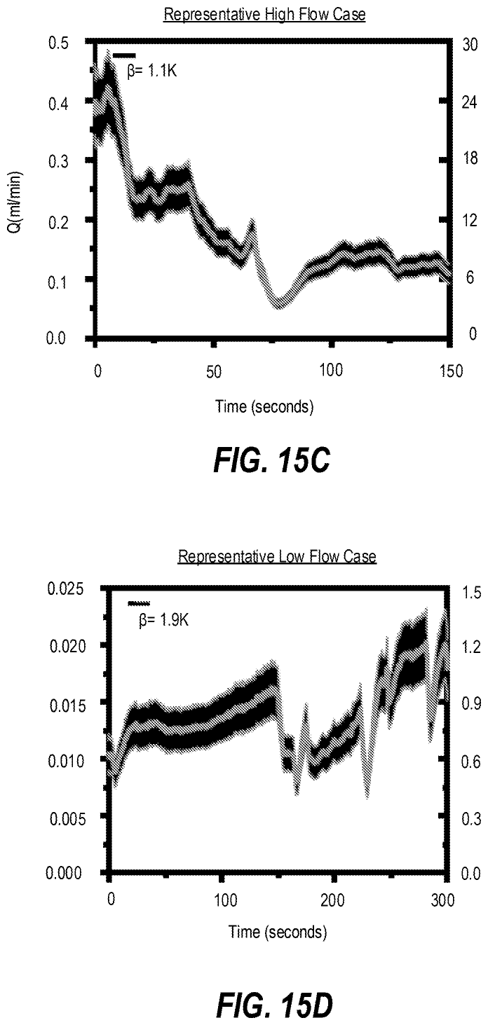

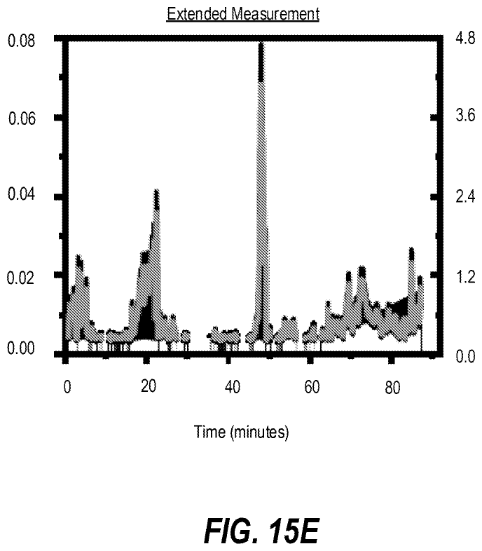

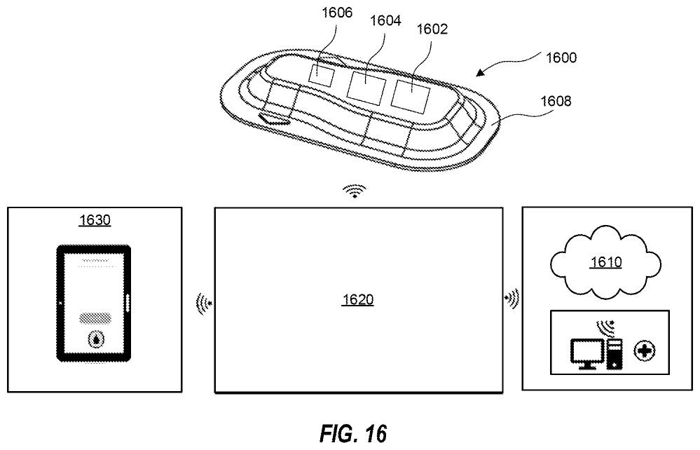

Reference is now made to FIGS. 14A-B and 15A-E, which illustrate finite element analysis (FEA) simulated data for α and β for hₛₖᵢₙ=1.4 mm corresponding to a flow rate of 0.07 ml/min, and simulated data for low-flow (<0.07 ml/min) and high-flow regimes (>0.07 ml/min), respectively. The real-time measurement of a quantitative value of the flow rate, beyond the metric γ, may represent a key capability. Accounting for hₛₖᵢₙ=1.4 mm via ultrasound imaging allows for 3D FEA models for α(Q) and β(Q), across a range of physiologically relevant CSF flow rates from 0.007 ml/min-1 ml/min (0.4 ml/hr-60 ml/hr). Other parameters in the model may include the thermal conductivity (kₛₖᵢₙ=0.3 W/m-K), density (ρₛₖᵢₙ=1050 Kg/m³) and heat capacity (C_{p,skin}=3500 J/Kg-K) of the skin, and corresponding properties for the shunt (kₛₕᵤₙₜ=0.21 W/m-K, ρₛₕᵤₙₜ=965 Kg/m³, C_{p,shunt}=1460 J/Kg-K). Values of α and β computed via 3D FEA are within acceptable levels of agreement (˜15%) with measured values on a benchtop shunt system configured to approximate the relevant anatomy (hₛₖᵢₙ=1.7 mm). The inflection point between high and low flow regimes is 0.07 ml/min, corresponding to β=1.3 K, as shown in FIG. 14B. Dividing α(Q) its low-flow and high-flow components and fitting each separately allows for conversion from a to Q. The low-flow regime is fitted via an exponential relationship (Q=0.0038e^{8.161a}), while the high-flow regime is fitted via a power law (Q=0.007α^{-2.12}), as shown in FIG. 16. The fits are in strong agreement with FEA models for 0.01K<α<0.5K. Representative high and low-flow cases computed in this manner, with their corresponding values of β are further illustrated in FIGS. 15A-E, where the shaded regions correspond to uncertainty estimates (15%) inherent to the fitting.

The results of 12 spot-check measurements performed on the same healthy outpatient over a 3-day period while sitting or standing upright are listed in Table 1. Averaging over 100 s windows yields values for β to define the high and low-flow regimes. Averaged values of α over the same window yield values for Q via the appropriate conversion equation, with standard deviations of ±15% associated with fitting uncertainties as the upper and lower bounds. The highest and lowest flow rates over the 3-day measurement are 0.26±0.05 ml/min (15.6±3 ml/h) and <0.01 ml/min (0.6 ml/h), respectively. Across the 12-measurements, 4 instances of high flow (Q>0.07 ml/min, β<1.3K), 6 instances of low-flow (Q<0.07 ml/min, β>1.3K), and 2 instances of transition-flow (Q˜0.07 ml/min, β˜1.3K) occurred. The average value of Q across all measurements was 0.08±0.07 ml/min (4.8±4.2 ml/hr). These flow rates correspond well to established values for pediatric and adult patients, across several studies on externalized drains and indwelling shunts. Flow varies significantly over a 60-minute period, consistent with current understanding of CSF hydrodynamics, and extended measurements on the same patient, as shown in FIGS. 15A-E. These data suggest the importance of either a single continuous measurement or several short measurements over a 60-minute period to accurately capture flow characteristics.

Table 1 illustrates flow rates calculated for 12 spot check measurements on a healthy out-patient across a 3-day period, with values of β for classification into high (β<1.3 K green), low (β>1.3 K, red) and transition (β˜1.3 K blue) flow regimes.

TABLE 1

| | Time of | | | Flow Rate |
|---|---|---|---|---|
| Day | Measurement | β (K) | Classification | (ml/min) |
| 1 | 12:57 PM | 1.12 ± 0.02 | High Flow | 0.14 ± 0.03 |
| 1 | 1:17 PM | 1.74 ± 0.04 | Low Flow | 0.01 ± 0.002 |
| 1 | 1:46 PM | 1.48 ± 0.03 | Low Flow | <0.01 |
| 1 | 2:01 PM | 1.64 ± 0.02 | Low Flow | 0.02 ± 0.007 |
| 1 | 2:45 PM | 1.70 ± 0.02 | Low Flow | 0.05 ± 0.02 |
| 2 | 1:36 PM | 1.12 ± 0.01 | High Flow | 0.12 ± 0.04 |
| 2 | 3:20 PM | 1.00 ± 0.04 | High Flow | 0.20 ± 0.04 |
| 2 | 4:08 PM | 1.65 ± 0.01 | Low Flow | 0.01 ± 0.003 |
| 2 | 5:00 PM | 1.11 ± 0.04 | High Flow | 0.26 ± 0.05 |
| 3 | 10:57 AM | 1.40 ± 0.03 | Transition | 0.06 ± 0.01 |
| 3 | 11:30 AM | 1.72 ± 0.01 | Low Flow | 0.01 ± 0.002 |
| 3 | 1:30 PM | 1.37 ± 0.01 | Transition | 0.08 ± 0.01 |

Reference is now made to FIG. 16, which illustrates a block diagram of a wireless fluid flow monitoring system, consistent with embodiments of the present disclosure. The system may include a wireless flexible flow sensor 1600 comprising a wireless receiver/transmitter 1602 to communicate with a controller, a flexible antenna 1604 for NFC based wireless charging, a three-axis digital accelerometer 1606, and a replaceable nonirritating adhesive surface 1608 for repeated and extended use. The system may further include a wireless charging station 1620, a storage database 1610, such as a cloud storage server, a network, and a microprocessor, among other components. In some embodiments, one or more components of the system may be wirelessly connected to each other. Although not illustrated, it is appreciated that some components of the fluid flow monitoring system may communicate using a wired connection.

In some embodiments, flow sensor 1600 disclosed herein may comprise a telemedicine-compatible flow sensing device configured to be used by outpatients at a non-hospital location such as home, office, etc. Telemedicine represents a critical means to deliver care for patients suffering from chronic conditions, particularly where unnecessary exposure of patients to hospital settings may be strongly discouraged. Moreover, the availability of high-quality home care to patients who do not have ready access to hospitals may enhance the treatment of patients suffering from chronic conditions such as hydrocephalus. Accordingly, the development of next-generation noninvasive, wearable sensors that can be worn and operated by patients at home represents a critical need for the development of remote care paradigms. The flow monitoring system, including the wireless, flexible flow sensor 1600 may be used by patients with shunted hydrocephalus, their family members, or the care providers. The system, as a whole, may also be used for pediatric and adult populations, where parents are expected to be the primary operators of the device for the former.

In some embodiments, the flexible flow sensor 1600 may be a reusable device configured to be reused several times a day, once every day, once every week, at predetermined time intervals during the day, or at any frequency, as appropriately needed or advised by a care provider. In such instances, the flow sensor may benefit from a rechargeable power source such as rechargeable battery, that can support a single long operation time (1-1.5 hours) or multiple shorter operation times (at least 20 minutes) adding up to 1.5-2 hours of measurement times per day. The flow sensor may comprise a near-field communication (NFC) based charging circuit including a flexible antenna configured to receive electromagnetic energy from a wireless charging station. In some embodiments, the charging circuit may include an inductive coil, an overcharge protection circuit, a capacitor, among other components useful for receiving, storing, and discharging the source, as needed. The overcharge protection circuit may comprise a Zener-diode based circuit configured to protect a battery from over-charging.

In some embodiments, wireless charging station 1620 may be configured to provide electrical charge to the flexible flow sensor by transmitting electromagnetic energy to the receiver/antenna of the charging circuit. The wireless charging station 1620 may be a stand-alone charger configured to receive or securely hold the flexible flow sensor 1600. In some embodiments, the flow sensor 1600 may be placed in proximity to the wireless charging station 1620 such that the flow sensor is within a transmission range of the wireless charging station 1620. In some embodiments, the wireless charging station may comprise a receptacle configured to receive and secure the flow sensor 1600 to minimize the time required to charge the flow sensor 1600.

The wireless fluid flow monitoring system may further comprise an electronic device such as a hand-held device 1630 (e.g., a tablet, a smartphone, a laptop, etc.) with a graphical user interface (GUI), in wireless communication with one or more of the flow sensor 1600, wireless charging station 1620, a database (not shown), a cloud storage server 1610, a network, among other things. The electronic device may comprise a microprocessor such as a ASIC chip, a VLSI chip, or any digital integrated circuit. The electronic device may further comprise a graphical user interface including, but not limited to, a display screen, a touch screen, an audio-visual interface, etc.

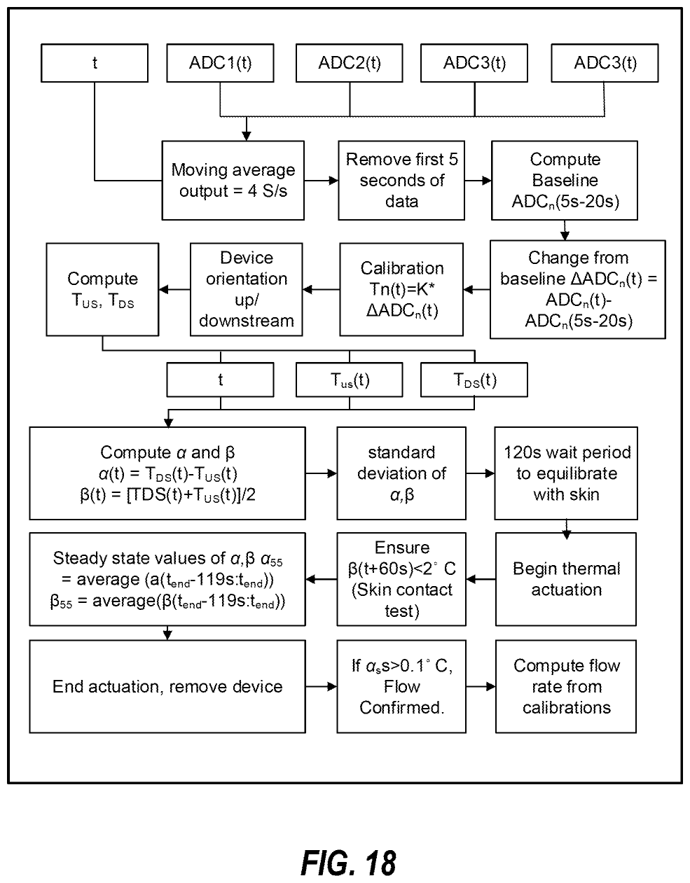

In some embodiments, electronic device 1630 may be configured to wirelessly communicate with the flow sensor 1600 and receive data associated with the flow of the fluid through the CSF shunt, for example. In some embodiments, the data may be received in real-time without prompting, upon prompting, or at predetermined time intervals. The microprocessor may be further configured to store and process the data received from the flow sensor 1600. For example, the microprocessor may perform data processing, that converts raw data streams into quantitative metrics of thermal anisotropy and flow. Wireless data transmission through cellular/WiFi networks may facilitate data transfer from the flow sensor device to the microprocessor of the hand-held device. The hand-held electronic device 1630 may upload this data to cloud storage server 1610 for remote access by patients and their attending physicians, at a later time. Accordingly, the device may be equipped with cellular and WiFi connectivity, with the ability to toggle between the two data modes to maximize data transfer rates and minimize user cost. In the first mode, unprocessed data may be transferred from the flow sensor 1600 to the microprocessor, and in the second mode, unprocessed or processed data may be transferred to a cloud storage server 1610, or an external database. In some embodiments, data upload to the cloud may also facilitate processing with advanced, machine-learning based algorithms (discussed later with reference to FIG. 18).

In some embodiments, a software application on the hand-held device (e.g., a laptop, or a smart phone) may communicate with the flow sensor 1600 directly and allow users to access data readout in real-time in a user-friendly format including, but not limited to, graphical representation, tabular representation, textual summary, comparative analysis charts, etc.

A GUI on the hand-held device may allow users to monitor basic device functions such as charging, operation, data upload and sleep mode, among other things. In some embodiments, the hand-held device may comprise memory to temporarily store data between upload events, for example. The range and connectivity of cellular and WiFi networks may allow the flow sensing device user to move around freely in their homes, or designated spaces while using the device, allowing them to transmit data from any location within a 100 m radius, for example.

In some embodiments, the flow sensor 1600 may comprise an accelerometer 1606 to determine and detect patient position such as if the patient is lying flat, sitting upright, inclined forward, reclined backwards, etc. Patient position may strongly influence CSF hydrodynamics. The flow sensor 1600 may comprise commercially available accelerometers, with materials, mechanics and integration schemes compatible with the rest of components of the flow sensor 1600 or the fluid flow monitoring system.

In some embodiments, flow sensor 1600 may comprise an adhesive layer 1608 capable of being facilely mounted onto the underside of the device layer for soft, nonirritating adhesion to a subject's skin. The adhesive layer 1608 may also provide strong adhesion of the fPCB substrate and other components to the elastomeric substrate.

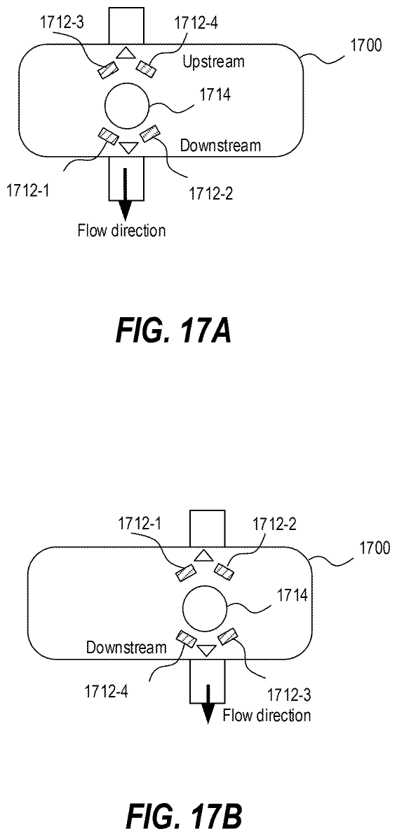

Reference is now made to FIGS. 17A and 17B, which is a schematic illustration of exemplary relative orientations of a thermal actuator and analog-to-digital converters in a wireless flow sensing system, consistent with disclosed embodiments. The flow sensor 1700 may comprise a thermal actuator 1714, and a pair of temperature sensors upstream 1712-3 and 1712-4 and temperature sensors downstream 1712-1 and 1712-2, in FIG. 17A of thermal actuator 1714, respectively, for a total of 4 temperature sensors. Negative Temperature Coefficient (NTC) temperature sensors form the basis of the temperature sensing circuit on the flow sensor device. An analog front-end circuit based on an amplified Wheatstone bridge circuit may be configured to convert changes in temperature induced NTC resistance into voltage signals. Analog digital converters (ADC) may digitize the voltage signals, to be wirelessly transmitted to a receiver of a hand-held device or a receiving unit of the flow sensor device itself. In some embodiments, in addition to the four ADC channels, the flow sensor device may include a fifth channel with a time stamp.

Negative temperature coefficient (NTC) temperature sensors may provide high accuracy and precision in measurements of temperature (<5 mK), with minimal hysteresis, good stability, and negligible drift. For redundancy, the flow sensor device may incorporate a pair of NTC elements upstream and downstream, for a total of 4 NTCs, located 1.5 mm from the edge of the actuator, for example. The measurement may involve delivery of thermal powers of 2-5 mW/mm² to the skin. In a typical configuration, the thermal actuator may exploit between 12 and 24 surface-mount resistors (300 μm×250 μm×600 μm) arranged in a dense, circular array over an area of 7.0 mm² to produce spatially uniform heating with a magnitude controlled by the applied voltage. As an example, a voltage of 3.3 V applied to a thermal actuator constructed with 20Ω resistors, for a total resistance of 24×20Ω=480Ω, results in a current of 7 mA and a power of P=23 mW, over an area of 7 mm² to yield a power density of 3.3 mW/mm². The result is a temperature increase of <5 K uniformly over the area of the actuator when mounted on skin or on a benchtop shunt phantom system.

As illustrated in FIG. 17A, in orientation 1 of the flow sensor 1700, temperature sensors 1712-1 and 1712-2 may be positioned downstream of thermal actuator 1714, and temperature sensors 1712-3 and 1712-4 may be positioned upstream of the thermal actuator 1714. The downstream direction refers to the direction of the CSF flow away from the ventricle into the peritoneal cavity. In orientation 2, as illustrated in FIG. 17B, temperature sensors 1712-1 and 1712-2 may be positioned upstream of the thermal actuator 1714 and temperature sensors 1712-3 and 1712-4 may be positioned downstream of the thermal actuator 1714. The orientation of the flow sensor device relative to the direction of flow may determine which pair of NTCs are classified as ‘upstream’ and ‘downstream’ respectively, and each pair may be averaged to yield a single upstream and downstream temperature measurement as a function of time.

Reference is now made to FIG. 18, which is a process flowchart outlining processing steps for flow conversion, determining aberrant skin contact, thermal equilibrium and signal conditioning. Additionally, or alternatively, the flowchart may comprise post-measurement processing steps including computing flow rate from calibrations, generating conclusory indications such as flow confirmed, etc.

Mounting the device onto the patient's skin may result in a thermal equilibration period (˜120 s) during which the NTCs attain body temperature. Following this step, a series of signal conditioning steps ensure data quality. The first 5 s of data may be discarded to omit any lingering thermal equilibration effects, after which the following 15 s of data are averaged to yield a baseline value. All subsequent temperature measurements are in relation to this baseline value. A simple calibration factor is used to convert the change in ADC value from its baseline (ΔADC(t)) into its corresponding change in temperature (ΔTn(t)). The orientation of the flow sensor device relative to the direction of flow may determine which pair of NTCs are classified as ‘upstream’ and ‘downstream’ respectively, and each pair is averaged to yield a single upstream and downstream temperature measurement as a function of time (T_{US}(t). T_{DS}(t)). Computing the average (β) and difference (α) between these two temperature data streams may result in quantitative metrics of flow. In some embodiments, α and β can be combined to yield a clinically useful relative flow measurement, and diagnostically important determinations of flow vs. no flow, curve fitting to finite element simulations can yield quantitative absolute flow rate values. This latter capability represents a first-in-kind diagnostic measure for patients with hydrocephalus. Additionally, the algorithm may incorporate checks for skin contact and thermal drift, with customized error messages guiding the user in the event of inaccurate device placement or device delamination. Finally, accelerometry data will be time-stamped with flow data to yield a single readout comprising flow as a function of time and patient position. The multistep signal processing and flow computation algorithm may be incorporated into an embedded firmware of the hand-held device (e.g., smartphone, tablet) for real-time readout. As described previously, this readout will also be accessible on the handheld tablet interface via real-time wireless transmission.

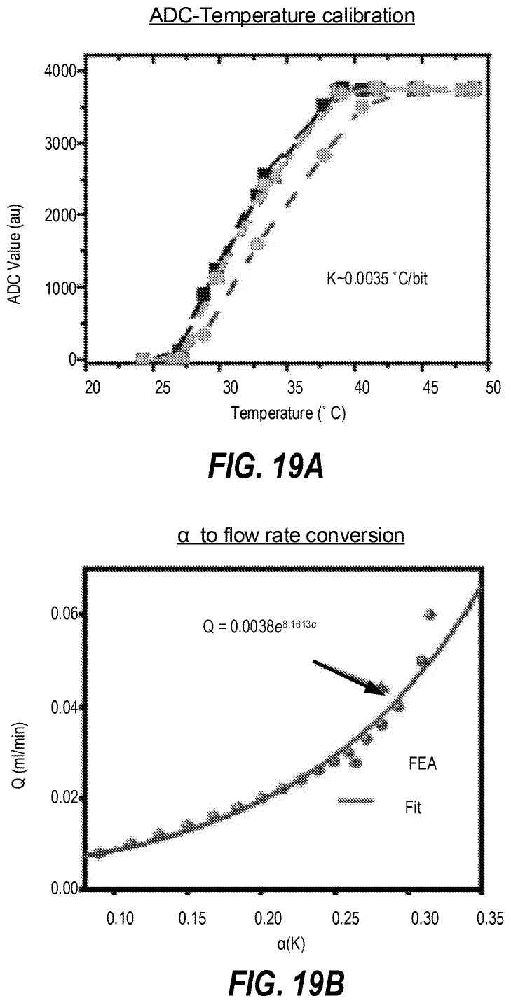

Reference is now made to FIG. 19, which illustrates data associated with ADC-temperature calibration and conversion of the temperature difference between upstream and downstream temperature sensors into flow rates of the fluid through the CSF shunt, consistent with some disclosed embodiments. As an example, the calibration factor that converts the change in ADC value from its baseline into a corresponding change in temperature may be 0.0035° C./bit, as shown in FIG. 19A. Based on the comparison with the FEA models and curve fitting, quantitative absolute flow rate values of the CSF flow may be determined, as illustrated in FIG. 19B.

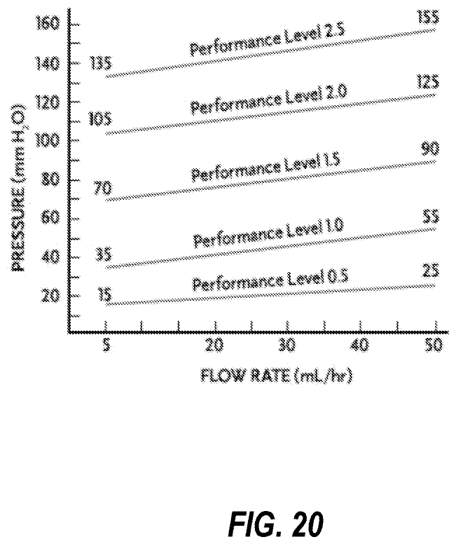

Reference is now made to FIG. 20, which illustrates an exemplary calibration chart for a commercially available ventriculoperitoneal (VP) shunt assembly valve. The commercially available valves to regulate CSF flow may be calibrated by the manufacturer, or may be adjusted by the end-user based on factory recommendations, patient's age, position, gender, medical condition, among other things. The calibration chart, relating ICP to CSF flow rate, may be used as a guide to estimate the ICP for a given range of CSF flow rate, for a given performance level. Thus, by measuring the CSF flow rate, one can approximately determine the ICP. As an example, for a performance level of 1.5, a CSF flow rate of 30 mL/hr may indicate an ICP of 80 mm H₂O.

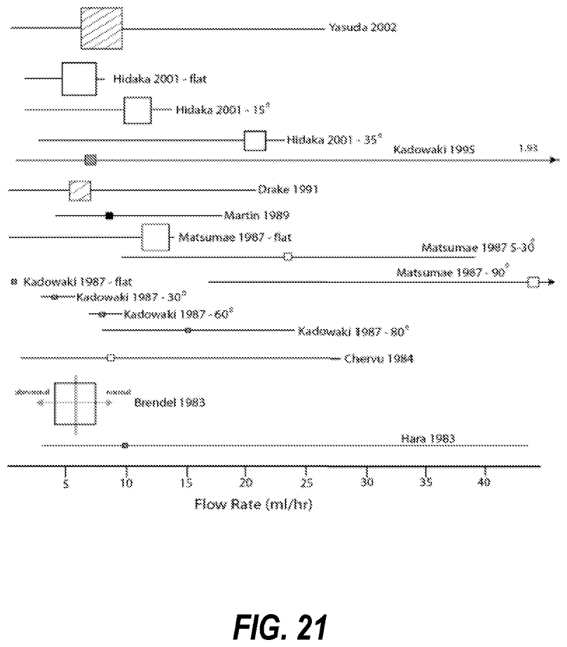

In some embodiments, CSF flow rate may depend on a number of factors including, but not limited to, a patient's age, supine position of the patient, time of day, among other factors. For example, as illustrated in FIG. 21, the average values of CSF flow rates across ages largely fall in the range of 5-30 mL/hr with surges noted for patients seated at approximately 90° with reference to a horizontal surface.

As described herein and in “Epidermal electronics for noninvasive, wireless, quantitative assessment of ventricular shunt function in patients with hydrocephalus,” Science Translational Medicine 10, eaat8437 (2018), “Wireless, Battery-free Epidermal Electronics for Continuous, Quantitative, Multimodal Thermal Characterization of Skin,” Small, 1803192 (2018). “Multimodal epidermal devices for hydration monitoring,” Microsystems & nanoengineering, 3, 1-11 (2017), “Continuous, noninvasive wireless monitoring of flow of cerebrospinal fluid through shunts in patients with hydrocephalus,” NPJ Digital Medicine 3, 1-11 (2020), all of which are incorporated by reference in their entireties, discuss systems and methods of quantitative measurement of CSF flow rates based on thermal anisotropy. Thermal actuators (miniaturized heating elements) may be configured to precisely dose controlled amounts of heat directly into the surface of skin overlying the implanted shunts. CSF underlying the actuator preferentially transports heat along its flow direction via convective effects, resulting in anisotropic thermal transport. This thermal anisotropy may be captured and measured via precise temperature sensors positioned upstream and downstream of the actuators along the direction of flow. The raw thermal anisotropy data from a relative flow rate (RFR) may be used to determine a true flow rate (TFR) (in conventional flow units such as mL/hr or mL/min). Constitutive and geometrical properties of the patient's skin that govern thermal transfer are essential unknowns in the direct conversion of temperature anisotropies to TFR.

### Thermal Diffusivity (ν) of Skin:

Thermal diffusivity is the ratio between thermal conductivity (k) and volumetric heat capacity (ρC, where ρ is density and C is specific heat capacity for constant pressure). This quantity can either be assumed to be known a priori based on extensive literature measurements to be k=0.32 W/m-K, ν=0.11 mm²/s, or measured based on transient plane source (TPS) techniques with the same sensor. In the latter approach, the sensor is placed on the clavicle adjacent to the shunt, in a region without any sources of anisotropy such as large blood vessels. A 60-300 s measurement in this region will allow for the measurement of thermal transport properties.

### Skin Thickness (h):

The thickness of skin between the actuation element and the underlying shunt is a critical parameter. As with thermal properties, there are two ways to address this unknown. In the first approach, the skin thickness may be assumed based on large (˜100) numbers of measurements of skin thickness overlying clavicle regions in patients with shunts using handheld ultrasound doppler tools. Data collected so far suggests an average skin thickness of h˜2 mm±0.5 mm over superficial clavicle regions.

In the second approach, a time-of-flight technique may be used. Thicker skin results in longer thermal diffusion distances and therefore longer times to equilibration. A systematic series of curves for skin thicknesses ranging from 0.5 mm<h<5 mm may be generated on realistic benchtop phantom assemblies, that simulate key geometrical and thermal features of the shunt-skin system. Modeling data suggests that skin thickness dominantly influences the time-constant, τ, to equilibration (defined as the time to 63.7% of the equilibrium temperature or temperature differential). Additionally, this is a parameter that is insensitive to flow rate. Generating these curves will provide simple lookup tables with which to compare in vivo data for the time constant, r as a measure of skin thickness, h.

### True Flow Rate (TFR) Measurement:

In some embodiments, the accurate measurements of thermal diffusivity of skin and skin thickness can allow for determining true flow rate (TFR) based on the relative flow rate (RFR) measurements. One of several ways to accomplish a TFR measurement based on RFR is by using algorithms based on benchtop data and modeling or simulation data. Thermal anisotropy (defined as ΔT=T_{Downstream}−T_{Upstream}) does not proceed monotonically with flow rate. ΔT has a peak value at ˜0.1 ml/min (6 mL/hr) where its sensitivity to flow is highest and declines on either side of this peak. Distinguishing degenerate points on either side of this curve relies on the knowledge of a second quantity, the average temperature T_{Avg}=(T_{Downstream}+T_{Upstream})/2 that varies inversely monotonically with flow and is more sensitive at higher flow rates. Once skin thickness is determined, based on either of the techniques listed above (a priori assumption or computation based on time-of-flight), iterative solving for ΔT and T_{average} can allow for determination of flow rate. The functional relationships for ΔT (TFR) and T_{average} (TFR) can either be experimentally determined based on realistic benchtop systems or computed based on 3D finite element models that are validated against benchtop measurements. Details of these experimental procedures are disclosed in “Epidermal electronics for noninvasive, wireless, quantitative assessment of ventricular shunt function in patients with hydrocephalus,” Science Translational Medicine, 10, eaat 8437 (2018), and “Continuous, noninvasive wireless monitoring of flow of cerebrospinal fluid through shunts in patients with hydrocephalus,” NPJ Digital Medicine, 3, 1-11 (2020), both of which are incorporated by reference in their entireties.

In some embodiments, the TFR may be determined based on the RFR through direct in-vivo calibrations based on external ventricular drains. This approach involves the direct generation of calibration curves to measure ΔT (TFR) and T_{average} (TFR) based on external ventricular drains on consenting patients. Sterilized silicone skin phantoms can be placed over EVD assemblies with varying thicknesses. Changing drainage bag height directly modulates pressure differential, and therefore, TFR. Measurements of ΔT (TFR) and T_{average} (TFR) in this context will allow for functional generation of curves that directly correlate pressure differentials with ΔT (TFR) and T_{average} (TFR) for a range of skin thicknesses, in a manner that can be used to generate calibration curves for direct measurements of TFR.

In some embodiments, the two approaches discussed above for TFR measurements can be used for measuring ICP. While the second approach involving direct in-vivo calibrations based on external ventricular drains may also allow for direct measurement of ICP without computation, the ability to correlate to valve settings will allow for a built-in system for data validation and testing. Experimental refinement may determine a set of parameters in measurement time, time of day and patient position that may optimize the ICP measurement.

It will be appreciated that the embodiments of the present disclosure are not limited to the exact construction that has been described above and illustrated in the accompanying drawings, and that various modifications and changes may be made without departing from the scope thereof. The present disclosure has been described in connection with various embodiments, other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The descriptions above are intended to be illustrative, not limiting. Thus, it will be apparent to one skilled in the art that modifications may be made as described without departing from the scope of the claims set out below.

What is claimed is:

1. A wireless, flexible flow sensor mountable on a body, the sensor comprising:
a substrate;
a thermal actuation mechanism supported by the substrate and configured to supply thermal energy to a portion of a skin surface of the body, the portion of the skin surface overlaying a subdermal conduit for a body fluid;
a temperature sensor supported by the substrate and configured to detect a change in a temperature related to the portion of the skin surface;
a motion sensor supported by the substrate and configured to detect an orientation related to a segment of the subdermal conduit;
a microprocessor in wireless communication with a controller, comprising circuitry configured to:
receive, from the controller, a first signal to activate the thermal actuation mechanism from the controller; and
receive, from the temperature sensor, a second signal associated with the change in temperature related to the portion of the skin surface; and
a power source configured to supply electrical power to at least one of the thermal actuation mechanism, the temperature sensor, and the microprocessor.

2. The sensor of claim 1, wherein the thermal actuation mechanism comprises a thermal actuator configured to receive the first signal from the controller.

3. The sensor of claim 1, wherein the temperature sensor comprises an upstream temperature sensor configured to detect a change in a temperature related to an upstream portion of the skin surface, and a downstream temperature sensor configured to detect a change in a temperature related to a downstream portion of the skin surface.

4. The sensor of claim 3, wherein the upstream and the downstream temperature sensors are positioned opposite to each other and separated by the thermal actuation mechanism.

5. The sensor of claim 1, wherein the temperature sensor comprises a thin film temperature sensor, a diode temperature sensor, a positive temperature coefficient of resistance (PTC) sensor, a negative temperature coefficient of resistance (NTC) sensor, a colorimetric temperature sensor, or a thermistor.

6. The sensor of claim 1, further comprising an adhesive layer in adhesive contact with a skin-facing surface of the substrate.

7. The sensor of claim 1, further comprising an encapsulation layer to encapsulate electronically active components supported by the substrate.

8. The sensor of claim 1, further comprising a thermal insulation layer to insulate the thermal actuation mechanism and the temperature sensor supported by the substrate.

9. The sensor of claim 1, wherein the power source comprises a rechargeable battery, a rechargeable Li-polymer battery, or a solid-state battery.

10. The sensor of claim 1, wherein the subdermal conduit comprises at least one of a blood vessel, a catheter, or a cerebrospinal fluid shunt, and wherein the body fluid comprises blood or cerebrospinal fluid.

11. A wireless fluid-flow monitoring system, comprising:
a flexible flow sensor mountable on a body, the flow sensor comprising:
a temperature sensor configured to continuously detect a change in temperature related to a portion of a skin surface of the body, the portion of the skin surface overlaying a subdermal conduit for a body fluid;
a power source configured to supply electrical power to at least one of the thermal actuation mechanism and the temperature sensor;
a receiving circuit in electrical communication with the power source, wherein the receiving circuit is configured to receive electromagnetic energy;
a power charging unit configured to wirelessly transmit electromagnetic energy to a receiver of the receiving circuit; and
a processor in wireless communication with the flexible flow sensor, the processor configured to:
receive, from the flexible flow sensor, data associated with the change in temperature related to the portion of the skin surface of the body:
determine a flow rate of the body fluid through a segment of the subdermal conduit based on the received data; and
store, in a database, the received data and the determined flow rate of the body fluid.

12. The system of claim 11, wherein the receiving circuit further comprises an overcharge protection circuit configured to modulate a power supply to the power source.

13. The system of claim 11, wherein the flexible flow sensor further comprises:
a motion sensor configured to detect an orientation related to a segment of the subdermal conduit;
a thermal actuation mechanism configured to supply thermal energy to the portion of the skin surface;
an adhesive layer in adhesive contact with a skin-facing surface of a substrate supporting the thermal sensor;
an encapsulation layer to encapsulate electronically active components supported by the substrate; and
a thermal insulation laver to insulate the thermal actuation mechanism and the temperature sensor supported by the substrate.

14. The system of claim 11, wherein continuous detection of the change in temperature related to the portion of the skin surface of the body comprises detection for a time period of at least 20 minutes.

15. The system of claim 11, further comprising a graphical user interface configured to display the stored data, wherein the graphical user interface comprises a touch screen, a visual display, or an audio-visual display.

16. The system of claim 11, wherein the processor is in wireless communication with the power charging unit.

17. A method of continuous flow measurement of a body fluid using a wireless, flexible flow sensor comprising a motion sensor, the method comprising:
sending, to a user-device for display, an indication to mount the flow sensor on a portion of a skin surface of a body, the portion of the skin surface overlaying a subdermal conduit of the body fluid;
detecting, using the motion sensor, a first position of the body;
sending, to the user-device for display, a second indication to adjust a position of the body to a second position of the body different from the first position;
detecting, using the motion sensor, the second position of the body; and
determining a change in flow related to the body fluid through a segment of the subdermal conduit, corresponding to a change related to the position of the body.

18. A computer-implemented system for continuously determining a flow rate of a body fluid through a subdermal conduit, the system comprising:
a memory storing instructions; and
a processor configured to execute the instructions to:
receive, from a temperature sensor of a flexible flow sensor, information associated with a temperature related to a portion of a skin surface, the portion of the skin surface overlaying the subdermal conduit, and a time of temperature measurement, the temperature sensor comprising:
a plurality of upstream temperature sensors configured to detect an upstream temperature related to the portion of the skin surface upstream of a thermal actuator of the flexible flow sensor;
a plurality of downstream temperature sensors configured to detect a downstream temperature related to the portion of the skin surface downstream of a thermal actuator of the flexible flow sensor:
receive, from the motion sensor, information associated with an orientation of a segment of the subdermal conduit and the time of temperature measurement;
compute a first value indicating a difference between the upstream and the downstream temperatures and a second value indicating an average of the upstream and downstream temperatures; and
determine the flow rate of the body fluid based at least in part on the computed first and second values.

19. The computer-implemented system of claim 18, wherein executing the instructions to determine the flow rate of the body fluid based at least in part on the computed first and second values comprises applying a model to the computed first and second values.

20. The computer-implemented system of claim 19, wherein executing the instructions to determine the flow rate of the body fluid based at least in part on the computed first and second values comprises applying the model to a plurality of the computed first and second values.

21. The computer-implemented system of claim 19, wherein applying the model comprises comparing at least one of the first and the second values to a database of values.

22. The computer-implemented system of claim 19, wherein applying the model comprises comparing at least one of the first and the second values to a lookup table.

23. The computer-implemented system of claim 19, wherein applying the model comprises comparing at least one of the first and the second values to a finite element analysis.

24. The computer-implemented system of claim 19, wherein applying the model comprises comparing at least one of the first and the second values to a set of previously measured first and second values.

25. The computer-implemented system of claim 18, wherein determining the flow rate of the body fluid based at least in part on the computed first and second values comprises determining an estimated flow rate or a range of the flow rate.

26. A method of measuring intracranial pressure using a wireless, flexible flow sensor assembly, the method comprising:
applying, using a thermal actuator of the flow sensor assembly, heat to a portion of a surface of a skin overlaying a subdermal conduit of the body fluid;
measuring, using a first thermal sensor of the flow sensor assembly, a first temperature of the body fluid flowing upstream of the thermal actuator;
measuring, using a second thermal sensor of the flow sensor assembly, a second temperature of the body fluid flowing downstream of the thermal actuator, determining a first flow rate related to the body fluid through a segment of the subdermal conduit based on a difference between the first and the second temperature of the body fluid;
determining a second flow rate related to the body fluid based on the first flow rate and a characteristic of the skin; and
determining intracranial pressure based on the determined second flow rate.

27. The method of claim 26, wherein the first flow rate comprises a relative flow rate of the body fluid, and wherein the second flow rate comprises a true flow rate of the body fluid.

28. The method of claim 26, wherein the characteristic of the skin comprises thermal diffusivity, or thickness of the skin.

29. The method of claim 26, further comprising determining a thermal anisotropy in the body fluid flowing through the subdermal conduit.
